SEQUENCE LISTING (17 sequences)

SEQ ID NO: 1 - protein, 10 aa, Artificial Sequence
  Description of Artificial Sequence Synthetic spacer peptide
IGKTISKKAK

SEQ ID NO: 2 - protein, 470 aa, Artificial Sequence
  Description of Artificial Sequence Humanized C2B8 Heavy Chain Sequence
MGWSLILLFLVAVATRVLSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 360 aa, Artificial Sequence
  Description of Artificial Sequence Humanized C2B8 Domain Deleted Heavy Chain Sequence
MGWSLILLFLVAVATRVLSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1413 bp, Artificial Sequence
  Description of Artificial Sequence Humanized C2B8 Heavy Chain Sequence
atgggttggagcctcatcttgctcttccttgtcgctgttgctacgcgtgtcctgtcccaggtacaactgcagcagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgcagacaaatcctccagcacagcctacatgcagctcagcagcctgacatctgaggactctgcggtctattactgtgcaagatcgacttactacggcggtgactggtacttcaatgtctggggcgcagggaccacggtcaccgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 5 - DNA, 1083 bp, Artificial Sequence
  Description of Artificial Sequence Humanized C2B8 Domain Deleted Heavy Chain Sequence
atgggttggagcctcatcttgctcttccttgtcgctgttgctacgcgtgtcctgtcccaggtacaactgcagcagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgcagacaaatcctccagcacagcctacatgcagctcagcagcctgacatctgaggactctgcggtctattactgtgcaagatcgacttactacggcggtgactggtacttcaatgtctggggcgcagggaccacggtcaccgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 6 - DNA, 708 bp, Artificial Sequence
  Description of Artificial Sequence Humanized C2B8 Light Chain Sequence
atggattttcaggtgcagattatcagcttcctgctaatcagtgcttcagtcataatgtccagaggacaaattgttctctcccagtctccagcaatcctgtctgcatctccaggggagaaggtcacaatgacttgcagggccagctcaagtgtaagttacatccactggttccagcagaagccaggatcctcccccaaaccctggatttatgccacatccaacctggcttctggagtccctgttcgcttcagtggcagtgggtctgggacttcttactctctcacaatcagcagagtggaggctgaagatgctgccacttattactgccagcagtggactagtaacccacccacgttcggaggggggaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 7 - protein, 235 aa, Artificial Sequence
  Description of Artificial Sequence Humanized C2B8 Light Chain Sequence
MDFQVQIISFLLISASVIMSRGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 354 aa, Artificial Sequence
  Description of Artificial Sequence Humanized HuCC49 Domain Deleted Heavy Chain Sequence
MGWSLILLFLVAVATRVLSQVQLVQSGAEVVKPGASVKISCKASGYTFTDHAIHWVKQNPGQRLEWIGYFSPGNDDFKYNERFKGKATLTADTSASTAYVELSSLRSEDTAVYFCTRSLNMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1065 bp, Artificial Sequence
  Description of Artificial Sequence Humanized HuCC49 Domain Deleted Heavy Chain Sequence
atgggttggagcctcatcttgctcttccttgtcgctgttgctacgcgtgtcctgtcccaggtccagctggtgcagtccggcgctgaggtggtgaaacctggggcttccgtgaagatttcctgcaaggcaagcggctacaccttcactgatcacgcaatccactgggtgaaacagaatcctggacagcgcctggagtggattggatatttctctcccggaaacgatgattttaagtacaatgagaggttcaagggcaaggccacactgactgcagacacatctgccagcactgcctacgtggagctctccagcctgagatccgaggatactgcagtgtacttctgcacaagatccctgaatatggcctactggggacagggaaccctggtcaccgtctccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 10 - protein, 240 aa, Artificial Sequence
  Description of Artificial Sequence Humanized HuCC49 Light Chain Sequence
MDSQAQVLMLLLLWVSGTCGDIVMSQSPDSLAVSLGERVTLNCKSSQSLLYSGNQKNYLAWYQQKPGQSPKLLIYWASARESGVPDRFSGSGSGTDFTLTISSVQAEDVAVYYCQQYYSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 723 bp, Artificial Sequence
  Description of Artificial Sequence Humanized HuCC49 Light Chain Sequence
atggatagccaggcccaggtgctcatgctcctgctgctgtgggtgagcggcacatgcggcgacatcgtgatgagccagtctccagactccctggccgtgtccctgggcgagagggtgactctgaattgcaagtccagccagtccctgctctatagcggaaatcagaagaactatctcgcctggtatcagcagaaaccagggcagagccctaaactgctgatttactgggcatccgctagggaatccggcgtgcctgatcgcttcagcggcagcggatctgggacagacttcactctgacaatcagcagcgtgcaggcagaagacgtggcagtctattattgtcagcagtattatagctatcccctcacattcggcgctggcaccaagctggaactgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 12 - protein, 468 aa, Artificial Sequence
  Description of Artificial Sequence Humanized C5E10 Heavy Chain Sequence
MAVLALLFCLVTFPSCILSQVQLKESGPGLVAPSQSLSITCTVSGFSLTDYGVNWVRQPPGKGLEWLGMIWDNGRTDYNSALKSRLSINKDNSKSQVFLKMTSLQTDDTARYYCARCYYGSSPYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 358 aa, Artificial Sequence
  Description of Artificial Sequence Humanized C5E10 Domain Deleted Heavy Chain Sequence
MAVLALLFCLVTFPSCILSQVQLKESGPGLVAPSQSLSITCTVSGFSLTDYGVNWVRQPPGKGLEWLGMIWDNGRTDYNSALKSRLSINKDNSKSQVFLKMTSLQTDDTARYYCARCYYGSSPYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 1407 bp, Artificial Sequence
  Description of Artificial Sequence Humanized C5E10 Heavy Chain Sequence
atggctgtcttagcgctactcttctgcctggtaacattcccaagctgtatcctttcccaggtgcagctgaaggagtcaggacctggcctggtggcgccctcacagagcctgtccatcacatgcaccgtctcagggttctcattaaccgactatggtgtaaactgggttcgccagcctccaggaaagggtctggagtggcttggaatgatatgggataatggaagaacagactataattcagctctcaaatccagactgagcatcaacaaggacaactccaagagccaagttttcttaaaaatgaccagtctgcaaactgatgacacagccaggtactactgtgccagatgctattacggtagtagcccttactttgactactggggccaaggcaccactctcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 15 - DNA, 1077 bp, Artificial Sequence
  Description of Artificial Sequence Humanized C5E10 Domain Deleted Heavy Chain Sequence
atggctgtcttagcgctactcttctgcctggtaacattcccaagctgtatcctttcccaggtgcagctgaaggagtcaggacctggcctggtggcgccctcacagagcctgtccatcacatgcaccgtctcagggttctcattaaccgactatggtgtaaactgggttcgccagcctccaggaaagggtctggagtggcttggaatgatatgggataatggaagaacagactataattcagctctcaaatccagactgagcatcaacaaggacaactccaagagccaagttttcttaaaaatgaccagtctgcaaactgatgacacagccaggtactactgtgccagatgctattacggtagtagcccttactttgactactggggccaaggcaccactctcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 16 - DNA, 717 bp, Artificial Sequence
  Description of Artificial Sequence Humanized C5E10 Light Chain Sequence
atgggcatcaagatggagtcacattctctggtctttgtatacatgttgctgtggttgtctggtgttgaaggagacattgtgatgatccagtctcacaaattcatgtccacatcagtaggagacagggtcagcatcacctgcaaggccagtcaggatgtgggtactgctgtcgcctggtatcaacagaaaccaggacaatctcctaaactactgatttactggtcatccacccggcacactggagtccctgatcgcttcacaggcagtggatctgggacagatttcactctcaccattagcaatgtgcagtctgaagacttggcagattatttctgtcagttatatagcagctatcctctcacgttcggaggggggaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 17 - protein, 238 aa, Artificial Sequence
  Description of Artificial Sequence Humanized C5E10 Light Chain Sequence
MGIKMESHSLVFVYMLLWLSGVEGDIVMIQSHKFMSTSVGDRVSITCKASQDVGTAVAWYQQKPGQSPKLLIYWSSTRHTGVPDRFTGSGSGTDFTLTISNVQSEDLADYFCQLYSSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2011/0020222 A1
(43) Pub. Date: Jan. 27, 2011

(54) MODIFIED ANTIBODIES AND METHODS OF USE

(75) Inventors: Gary R. Braslawsky, San Diego, CA (US); Nabil Hanna, Rancho Santa Fe, CA (US); Paul Chinn, Carlsbad, CA (US)

Correspondence Address: NELSON MULLINS RILEY & SCARBOROUGH / BIOGEN;FLOOR 30, SUITE 3000, ONE POST OFFICE SQUARE, BOSTON, MA, 02109-2127, US

(73) Assignee: BIOGEN IDEC MA Inc., Cambridge, MA (US)

(21) Appl. No.: 12/789,955

(22) Filed: May 28, 2010

Related U.S. Application Data
(63) Continuation of application No. 12/032,424, filed on Feb. 15, 2008, now abandoned.
Application No. 12/032,424 is a continuation of application No. 10/058,120, filed on Jan. 29, 2002, now abandoned.
(60) Provisional application No. 60/264,318, filed on Jan. 29, 2001.
(60) Provisional application No. 60/331,481, filed on Nov. 16, 2001.

(51) Int. Cl. A61K 51/10; C07K 16/30; A61K 49/00; A61K 39/395; A61P 35/00

(52) U.S. Cl. 424/1.49; 530/389.7; 530/391.7; 530/391.3; 424/9.1; 424/1.53; 424/133.1; 424/178.1

(57) ABSTRACT

Novel compounds, compositions and methods comprising modified antibodies are provided. In preferred embodiments the disclosed modified antibodies comprise antibodies having one or more of the constant region domains altered or deleted to afford beneficial physiological properties such as enhanced target localization and rapid blood clearance. The disclosed compounds are particularly useful for the treatment of neoplastic disorders in myelosuppressed patients.

### CROSS REFERENCE TO RELATED APPLICATIONS

This application is a continuation-in-part of U.S. Provisional Application No. 60/264,318 filed Jan. 29, 2001, and claims priority to U.S. Provisional Application No. 60/331,481 filed Nov. 16, 2001 each of which is incorporated in its entirety herein by reference.

### FIELD OF THE INVENTION

In a broad aspect the present invention relates to improved compositions and methods comprising modified immunoglobulins for the treatment of neoplastic disorders. More particularly, the present invention comprises the use of modified immunoglobulins exhibiting improved tumor localization and superior physiological profiles for the immunotherapeutic treatment of malignancies. The disclosed methods and compositions are especially useful in the treatment of cancer patients that are myelocompromised due to exposure to chemotherapeutic agents, external radiation or radioimmunotherapeutics.

### BACKGROUND OF THE INVENTION

Patients afflicted with relatively diverse malignancies have benefited from advances in cancer treatments over the past several decades. Unfortunately, while modern therapies have substantially increased remission rates and extended survival times, most patients continue to succumb to their disease eventually. Barriers to achieving even more impressive results comprise tumor-cell resistance and the unacceptable toxicity (e.g. myelotoxicity) of available treatments that limit optimal cytotoxic dosing and often make current therapies unavailable to immunocompromised, debilitated or older patients. These limitations are particularly evident when attempting to care for patients that have undergone previous treatments or have relapsed. Thus, it remains a challenge to develop less toxic, but more effective, targeted therapies.

One attempt at enhancing the effectiveness of such treatments involves the use of therapeutic antibodies to reduce undesirable cross-reactivity and increase tumor cell localization of one or more cytotoxic agents. The idea of recruiting antibodies to use in treating neoplastic disorders dates to at least 1953 when it was shown that antibodies could be used to specifically target tumor cells. However, it was the seminal work of Kohler and Milstein in hybridoma technology that allowed for a continuous supply of monoclonal antibodies that specifically target a defined antigen. By 1979, monoclonal antibodies (MAbs) had been used to treat malignant disorders in human patients. More recently three unconjugated monoclonal antibodies, Rituxan® Campath® & Herceptin®, have been approved for the treatment of non-Hodgkins lymphoma, CLL and breast cancer respectively. Currently, a number of monoclonal antibodies conjugated to various cytotoxic agents (e.g. radioisotopes or protein toxins) are in clinical trials related to the treatment of various malignant disorders. Over the past decade, a wide variety of tumor-specific antibodies and antibody fragments have been developed, as have methods to conjugate the antibodies to drugs, toxins, radionuclides or other agents, and to administer the conjugates to patients. These efforts have produced shown promise, but a variety of largely unanticipated problems have limited the diagnostic and therapeutic utility of some of the reagents thus far developed.

Among the most intractable problems is that which is caused by the human immune system itself, which may respond to the targeting conjugate as a foreign antigen. For instance, patients treated with drugs or radionuclides complexed with murine monoclonal antibodies (which have been the most commonly used targeting antibodies for human) develop circulating human anti-mouse antibodies (HAMAs) and a generalized immediate type-III hypersensitivity reaction to the antibody moiety of the conjugate. Furthermore, even when adverse side effects are minimal (for example, as in a single administration), circulating HAMAs decrease the effective concentration of the targeting agent in the patient and therefore limiting the diagnostic or therapeutic agent from reaching the target site.

Various problems continue to limit the clinical usefulness of RIT. Most commonly, the dosing of radiolabeled MAb immunotherapy (RIT) is limited by myelotoxicty through exposure of the circulating radiolabeled immunoconjugate (IC) to normal hematological cells residing in the red marrow. Patients who have previously undergone traditional chemotherapy are especially vulnerable through reduced red marrow reserves due to the extensive prior drug therapy. This has limited the use of RIT in combination with cytotoxic drugs, many of which are known to synergies the anti-tumor response of irradiated tumor cells. For example, it has been demonstrated that administration of ¹³¹I labeled anti-CEA MAb in combination with doxorubicin increases the therapeutic effect of the individual agents in a murine xenograft model of lung carcinoma. However the combination was more toxic than each component administered separately. Similar results were obtained using RIT in combination with cisplatin. Other drugs shown to synergize with RIT include, but are not limited to: metabolic enzyme inhibitors (e.g. MTX, Tomudex,) including Topisomerase enzyme inhibitors (podohylotoxins e.g. etoposide), anti-metabolites (e.g. fluorouracil), Porphyrin (gadolinium-texaphyrin) or DNA intercolators (e.g. Anthracyclins, Camptothecins etc).

Additionally, cancer patients having extensive bone marrow metastasis are especially at risk due to the additional irradiation of the red marrow via neighboring tumor cells that were targeted by the radiolabeled IC. As an example, Non-Hodgkin's lymphoma (NHL) patients treated with yttrium labeled Zevalin or ¹³¹I labeled Bexxar and chronic lymphocytic leukemia (CLL) patients treated with Lym-1, who have significant bone marrow metastases, are more likely to develop dose-limiting toxicity than patients without bone marrow involvement. Therefore further increasing the risk of myelotoxicity in these patient populations when used in combination with cytotoxic drug therapy.

One way to increase the therapeutic effectiveness of RIT would be to increase the dose of administered RIT thereby increasing the amount of isotope delivered or targeted via the MAb to the tumor. Previous studies have used enzymatically digested or genetically engineered MAb fragments that retain high affinity binding to the targeted cancer cell and are rapidly cleared from the blood to lower toxicity to the bone marrow. Examples include both monovalent (e.g. scFv and Fab fragments) and multivalent (e.g. F(ab′)₂, inverted F(ab′)₂ and double chain Fv fragments) antibody fragments. These constructs when compared to traditional ICs have demonstrated rapid clearance from blood in both murine animal models and human clinical trial. Reduced red marrow radiation exposure and a lower level of toxicity accompanied rapid blood clearance. Unfortunately, such constructs were also cleared from the tumor faster than traditional intact MAbs and were less efficient in their ability to target isotope to the tumor population. Thus, any potential advantage of using the faster blood clearance rate and lower toxicity of MAb fragments for combination therapy with anti-cancer drugs was offset by their inability to efficiently target isotope to the tumor site.

As such, it is an object of the present invention to provide low toxicity compounds that may be used to target neoplastic cells.

It is another object of the invention to provide compounds that may effectively used to treat myelosuppressed patients.

### SUMMARY OF THE INVENTION

These and other objectives are provided for by the present invention which, in a broad sense, is directed to methods, compounds and compositions that may be used in the treatment of neoplastic disorders. To that end, the present invention provides for modified antibodies that may be used to treat patients suffering from a variety of cancers. In this respect, the modified antibodies or immunoglobulins of the present invention have been surprisingly found to exhibit biochemical characteristics that make them particularly useful for the treatment of myelosuppressed patients. More specifically, it was unexpectedly found that the modified antibodies described herein are rapidly cleared from the blood while providing for effective tumor localization. As such, the disclosed compounds may be used to substantially reduce the toxicity associated with the non-specific dissemination of conventional immuno conjugates while still providing therapeutically effective levels of the selected cytotoxin at the site of the tumor. This is particularly true when the modified antibodies are used as radioimmunoconjugates.

Accordingly, one important aspect of the present invention comprises the use of the modified antibodies as radioimmunoconjugates to treat neoplastic disorders. That is, the modified antibody may be associated with a therapeutic radioisotope such as ⁹⁰Y or ¹³¹I and administered to patients suffering from any one of a number of cancers. The surprising properties of the disclosed compounds (i.e. rapid blood clearance and effective tumor localization) substantially reduces associated toxicity to healthy organs (especially the marrow) while delivering therapeutically effective doses directly to the tumor. This exhibited reduction in myelotoxicity makes the present invention particularly useful in the treatment of patients that are myelosuppressed or otherwise myelocompromised.

Quite often, myelosuppression is seen as a side effect of chemotherapeutic treatments such as radiation or the administration of toxic agents. As such, another significant aspect of the present invention is the use of the disclosed compounds (with or without an associated radioisotope) in conjunction with adjunct chemotherapy or radiation. It is particularly useful in patients that have relapsed or otherwise gone through prior chemotherapy resulting in a myelosuppressive state. In such patients (and often in relatively healthy patients) the dose limiting toxicity of radiolabeled antibodies is myelotoxicity through the exposure of circulating radioisotope to normal marrow cells. The present invention reduces this exposure and corresponding toxicity thereby allowing more efficacious and higher doses to be administered. However, unlike prior art compounds that reduce toxicity, the modified antibodies of the present invention still exhibit effective tumor localization thus further increasing the benefit to the patient.

It will further be appreciated that these same properties make the compounds and compositions of the present invention particularly suitable for diagnostic procedures such as radioimaging of tumors. That is, the modified antibodies of the present invention could be associated with diagnostic radioisotopes (i.e. ¹¹¹In) and used for the diagnosis or monitoring of neoplastic or other disorders. In this regard the rapid clearance of the unbound modified antibodies and the high and rapid tumor localization will provide for enhanced images having substantially better signal to noise ratios that those provided using conventional radioimaging agents. Of course those skilled in the art could easily determine which types of imaging (e.g. MRI, radioimaging, ultrasound, etc) and what particular imaging agents could be used effectively with the compounds disclosed herein.

Other objects, features and advantages of the present invention will be apparent to those skilled in the art from a consideration of the following detailed description of preferred exemplary embodiments thereof.

### BRIEF DESCRIPTION OF THE FIGURES

FIGS. 1A and 1B show, respectively, an amino acid sequence of an intact C2B8 heavy chain and an amino acid sequence of a derived domain deleted C2B8 construct wherein the C_{H}2 domain has been deleted;

FIGS. 2A and 2B show, respectively, a nucleotide sequence of an intact C2B8 heavy chain and a nucleotide sequence of a derived domain deleted C2B8 construct wherein the C_{H}2 domain has been deleted;

FIGS. 3A and 3B show, respectively, a nucleotide sequence of a C2B8 light chain and the corresponding amino acid sequence of the same light chain;

FIGS. 4A and 4B show, respectively, the amino acid sequence of a huCC49 domain deleted heavy chain wherein the C_{H}2 domain has been deleted and a corresponding nucleotide sequence for the same heavy chain;

FIGS. 5A and 5B show, respectively, an amino acid sequence of a huCC49 light chain and a corresponding nucleotide sequence of the same light chain;

FIGS. 6A and 6B show, respectively, an amino acid sequence of an intact C5E10 heavy chain and an amino acid sequence of a derived domain deleted C5E10 construct wherein the C_{H}2 domain has been deleted;

FIGS. 7A and 7B show, respectively, a nucleotide sequence of an intact C5E10 heavy chain and a nucleotide sequence of a derived domain deleted C5E10 construct wherein the C_{H}2 domain has been deleted;

FIGS. 8A and 8B show, respectively, a nucleotide sequence of a C5E10 light chain and the corresponding amino acid sequence of the same light chain;

FIG. 9 is a graphical representation of the blood clearance rates of intact huCC49 and huCC49.ΔC_{H}2 labeled with various radioisotopes in LS147T tumor bearing mice;

FIGS. 10A, 10B and 10C are, respectively, graphical representations of blood clearance and tumor localization rates of radiolabeled intact C2B8, C2B8.F(ab′)2 and C2B8.ΔC_{H}2 as determined in Daudi (CD20+) tumor murine xenograft models;

FIG. 11 illustrates the synergistic properties provided by a combination of radiolabeled huCC49.ΔC_{H}2 and etoposide in comparison with the use of the antineoplastic agents individually.

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated.

The present invention is predicated, at least in part, on the fact that antibodies which are immunoreactive with antigens associated with neoplastic cells may be modified or altered to provide enhanced biochemical characteristics and improved efficacy when used in therapeutic protocols on myelosuppressed patients. Preferably, the modified antibodies will be associated with a cytotoxic agent such as a radionuclide or antineoplastic agent. In this regard, it has surprisingly been found that antibodies modified according to the present invention may advantageously be used to provide radioimmunotherapy to patients having reduced red marrow reserves. More particularly, the modified antibodies of the present invention appear to exhibit more efficient tumor localization and a shorter serum half-life relative to whole antibodies having the same binding specificity. As such, they are particularly useful in targeting a cytotoxin such as a radionuclide to a malignant cell or tumor while minimizing unwanted exposure to healthy cells (e.g., hematologic cells). This increased efficacy allows for the more aggressive treatment of malignancies in myelosuppressed patients such as those who have previously undergone, or are currently undergoing, chemotherapy.

As used herein the term “modified antibody” shall be held to mean any antibody, or binding fragment or recombinant thereof, immunoreactive with a tumor associated antigen in which at least a fraction of one or more of the constant region domains has been deleted or otherwise altered so as to provide desired biochemical characteristics such as increased tumor localization or reduced serum half-life when compared with a whole, unaltered antibody of approximately the same binding specificity. In preferred embodiments, the modified antibodies of the present invention have at least a portion of one of the constant domains deleted. For the purposes of the instant disclosure, such constructs shall be termed “domain deleted.” Preferably, one entire domain of the constant region of the modified antibody will be deleted and even more preferably the entire C_{H}2 domain will be deleted. As will be discussed in more detail below, each of the desired variants may readily be fabricated or constructed from a whole precursor or parent antibody using well known techniques.

Those skilled in the art will appreciate that the compounds, compositions and methods of the present invention are useful for treating any neoplastic disorder, tumor or malignancy that exhibits a tumor associated antigen. As discussed above, the modified antibodies of the present invention are immunoreactive with one or more tumor associated antigens. That is, the antigen binding portion (i.e. the variable region or immunoreactive fragment or recombinant thereof) of the disclosed modified antibodies binds to a selected tumor associated antigen at the site of the malignancy. Given the number of reported tumor associated antigens, and the number of related antibodies, those skilled in the art will appreciate that the presently disclosed modified antibodies may therefore be derived from any one of a number of whole antibodies. More generally, modified antibodies useful in the present invention may be obtained or derived from any antibody (including those previously reported in the literature) that reacts with a tumor associated antigen. Further, the parent or precursor antibody, or fragment thereof, used to generate the disclosed modified antibodies may be murine, human, chimeric, humanized, non-human primate or primatized. In other preferred embodiments the modified antibodies of the present invention may comprise single chain antibody constructs (such as that disclosed in U.S. Pat. No. 5,892,019 which is incorporated herein by reference) having altered constant domains as described herein. Consequently, any of these types of antibodies modified in accordance with the teachings herein is compatible with the instant invention.

As used herein, “tumor associated antigens” means any antigen which is generally associated with tumor cells, i.e., occurring at the same or to a greater extent as compared with normal cells. More generally, tumor associated antigens comprise any antigen that provides for the localization of immunoreactive antibodies at a neoplastic cell irrespective of its expression on non-malignant cells. Such antigens may be relatively tumor specific and limited in their expression to the surface of malignant cells or showing increases in cell surface expression on malignant population when compared with non-malignant tissues. MAbs reactive with CEA, MUC-1 and TAG-72 are examples. Alternatively, such antigens may be constitutively expressed on both malignant and non-malignant cells. For example, CD20 is a pan B antigen that is found on the surface of both malignant and non-malignant B cells that has proved to be an extemely effective target for immunotherapeutic antibodies for the treatment of non-Hodgkin's lymphoma. In this respect, pan T cell antigens such as CD2, CD3, CD5, CD6 and CD7 also comprise tumor associated antigens within the meaning of the present invention. Other exemplary tumor associated antigens comprise but are not limited to MAGE-1, MAGE-3, HPV 16, HPV E6 & E7, L6-Antigen, CD19, CD22, CD37, HLA-DR, EGF Receptor and HER2 Receptor. In many cases immunoreative antibodies for each of these antigens have been reported in the literature. Those skilled in the art will appreciate that each of these antibodies may serve as a precursor for modified antibodies in accordance with the present invention.

The modified antibodies of the present invention preferably associate with, and bind to, tumor associated antigens as described above. Accordingly, as will be discussed in some detail below the modified antibodies of the present invention may be derived, generated or fabricated from any one of a number of antibodies that react with tumor associated antigens. In preferred embodiments the modified antibodies will be derived using common genetic engineering techniques whereby at least a portion of one or more constant region domains are deleted or altered so as to provide the desired biochemical characteristics such as reduced serum half-life. More particularly, as will be exemplified below, one skilled in the art may readily isolate the genetic sequence corresponding to the variable and/or constant regions of the subject antibody and delete or alter the appropriate nucleotides to provide the modified antibodies of the instant invention. It will further be appreciated that the modified antibodies may be expressed and produced on a clinical or commercial scale using well-established protocols.

In selected embodiments, modified antibodies useful in the present invention will be derived from known antibodies to tumor associated antigens. This may readily be accomplished by obtaining either the nucleotide or amino acid sequence of the parent antibody and engineering the modifications as discussed herein. For other embodiments it may be desirable to only use the antigen binding region (e.g., variable region or complementary determining regions) of the known antibody and combine them with a modified constant region to produce the desired modified antibodies. Compatible single chain constructs may be generated in a similar manner. In any event, it will be appreciated that the antibodies of the present invention may also be engineered to improve affinity or reduce immunogenicity as is common in the art. For example, the modified antibodies of the present invention may be derived or fabricated from antibodies that have been humanized or chimerized. Thus, modified antibodies consistent with present invention may be derived from and/or comprise naturally occurring murine, primate (including human) or other mammalian monoclonal antibodies, chimeric antibodies, humanized antibodies, primatized antibodies, bispecific antibodies or single chain antibody constructs as well as immunoreactive fragments of each type.

As alluded to above, previously reported antibodies that react with tumor associated antigens may be altered as described herein to provide the modified antibodies of the present invention. Exemplary antibodies that may be used to provide antigen binding regions for, generate or derive the disclosed modified antibodies include, but are not limited to Y2B8 and C2B8 (Zevalin™ & Rituxan®, IDEC Pharmaceuticals Corp., San Diego), Lym 1 and Lym 2 (Techniclone), LL2 (Immunomedics Corp., New Jersey), HER2 (Herceptin®, Genentech Inc., South San Francisco), B1 (Bexxar®, Coulter Pharm., San Francisco), MB1, BH3, B4, B72.3 (Cytogen Corp.), CC49 (National Cancer Institute) and 5E10 (University of Iowa). In preferred embodiments, the modified antibodies of the present invention will bind to the same tumor associated antigens as the antibodies enumerated immediately above. In particularly preferred embodiments, the modified antibodies will be derived from or bind the same antigens as Y2B8, C2B8, CC49 and C5E10 and, even more preferably, will comprise domain deleted antibodies (i.e., ΔC_{H}2 antibodies). As will be seen in the discussion and examples below, such modified antibodies are particularly useful the treatment of myelosuppressed patients or for use in conjunction with chemotherapy.

In a first preferred embodiment, the modified antibody will bind to the same tumor associated antigen as Rituxan®. Rituxan (also known as Rituximab, IDEC-C2B8 and C2B8) was the first FDA-approved monoclonal antibody for treatment of human B-cell lymphoma (see U.S. Pat. Nos. 5,843,439; 5,776,456 and 5,736,137 each of which is incorporated herein by reference). Y2B8 is the murine parent of C2B8. Rituxan is a chimeric, anti-CD20 monoclonal antibody (MAb) which is growth inhibitory and reportedly sensitizes certain lymphoma cell lines for apoptosis by chemotherapeutic agents in vitro. The antibody efficiently binds human complement, has strong FcR binding, and can effectively kill human lymphocytes in vitro via both complement dependent (CDC) and antibody-dependent (ADCC) mechanisms (Reff et al., Blood 83: 435-445 (1994)). Those skilled in the art will appreciate that variants of C2B8 or Y2B8, modified according to the instant disclosure, may be used in conjugated or unconjugated forms to effectively treat patients presenting with CD20+ malignancies. More generally, it will be appreciated that the modified antibodies disclosed herein may be used in either a “naked” or unconjugated state or conjugated to a cytotoxic agent to effectively treat any one of a number of neoplastic disorders.

In other preferred embodiments of the present invention, the modified antibody will be derived from, or bind to, the same tumor associated antigen as CC49. As previously alluded to, CC49 binds human tumor associated antigen TAG-72 which is associated with the surface of certain tumor cells of human origin, specifically the LS174T tumor cell line. LS174T [American Type Culture Collection (herein ATCC) No. CL 188] is a variant of the LS180 (ATCC No. CL 187) colon adenocarcinoma line.

It will further be appreciated that numerous murine monoclonal antibodies have been developed which have binding specificity for TAG-72. One of these monoclonal antibodies, designated B72.3, is a murine IgG1 produced by hybridoma B72.3 (ATCC No. HB-8108). B72.3 is a first generation monoclonal antibody developed using a human breast carcinoma extract as the immunogen (see Colcher et al., Proc. Natl. Acad. Sci. (USA), 78:3199-3203 (1981); and U.S. Pat. Nos. 4,522,918 and 4,612,282 each of which is incorporated herein by reference). Other monoclonal antibodies directed against TAG-72 are designated “CC” (for colon cancer). As described by Schlom et al. (U.S. Pat. No. 5,512,443 which is incorporated herein by reference) CC monoclonal antibodies are a family of second generation murine monoclonal antibodies that were prepared using TAG-72 purified with B72.3. Because of their relatively good binding affinities to TAG-72, the following CC antibodies have been deposited at the ATCC, with restricted access having been requested: CC49 (ATCC No. HB 9459); CC 83 (ATCC No. HB 9453); CC46 (ATCC No. HB 9458); CC92 (ATTCC No. HB 9454); CC30 (ATCC No. HB 9457); CC11 (ATCC No. 9455); and CC15 (ATCC No. HB 9460). U.S. Pat. No. 5,512,443 further teaches that the disclosed antibodies may be altered into their chimeric form by substituting, e.g., human constant regions (Fc) domains for mouse constant regions by recombinant DNA techniques known in the art. Besides disclosing murine and chimeric anti-TAG-72 antibodies, Schlom et al. have also produced variants of a humanized CC49 antibody as disclosed in PCT/US99/25552 and single chain constructs as disclosed in U.S. Pat. No. 5,892,019 each of which is also incorporated herein by reference. Those skilled in the art will appreciate that each of the foregoing antibodies, constructs or recombinants, and variations thereof, may be modified and used in accordance with the present invention.

Besides the anti-TAG-72 antibodies discussed above, various groups have also reported the construction and partial characterization of domain-deleted CC49 and B72.3 antibodies (e.g., Calvo et al. Cancer Biotherapy, 8(1):95-109 (1993), Slavin-Chiorini et al. Int. J. Cancer 53:97-103 (1993) and Slavin-Chiorini et al. Cancer. Res. 55:5957-5967 (1995)). It will be appreciated that the disclosed constructs provide modified antibodies that are compatible with the methods and compositions of the present invention. Yet, while the cited references showed that the clearance time of the domain deleted constructs was accelerated when compared to the whole parent antibodies, they fail to suggest that the disclosed constructs would prove particularly effective in treating myelosuppressed patients that had undergone or were undergoing chemotherapy as taught by the instant application. Rather, these references seem to suggest that rapid clearance of the constructs would make them particularly useful for diagnostic procedures rather than combined therapeutic regimens as provided for in the present invention.

Still other preferred embodiments of the present invention comprise modified antibodies that are derived from or bind to the same tumor associated antigen as C5E10. As set forth in copending application U.S. Pat. No. 6,207,805, C5E10 is an antibody that recognizes a glycoprotein determinant of approximately 115 kDa that appears to be specific to prostate tumor cell lines (e.g. DU145, PC3, or ND1). Thus, in conjunction with the present invention, modified antibodies (e.g. C_{H}2 domain-deleted antibodies) that specifically bind to the same tumor associated antigen recognized by C5E10 antibodies could be produced and used in a conjugated or unconjugated form for the treatment of neoplastic disorders. In particularly preferred embodiments, the modified antibody will be derived or comprise all or part of the antigen binding region of the C5E10 antibody as secreted from the hybridoma cell line having ATCC accession No. PTA-865. The resulting modified antibody could then be conjugated to a radionuclide as described below and administered to a patient suffering from prostate cancer in accordance with the methods herein.

In addition to the antibodies discussed above, it may be desirable to provide modified antibodies derived from or comprising antigen binding regions of novel antibodies generated using immunization coupled with common immunological techniques. Using art recognized protocols, antibodies are preferably raised in mammals by multiple subcutaneous or intraperitoneal injections of the relevant antigen (e.g., purified tumor associated antigens or cells or cellular extracts comprising such antigens) and an adjuvant. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood to provide homogenous preparations of monoclonal antibodies (MAbs). Preferably, the lymphocytes are obtained from the spleen.

In this well known process (Kohler et al., Nature, 256:495 (1975)) the relatively short-lived, or mortal, lymphocytes from a mammal which has been injected with antigen are fused with an immortal tumor cell line (e.g. a myeloma cell line), thus producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They therefore produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by immunoprecipitation or by an in vitro assay, such as a radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, pp 59-103 (Academic Press, 1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

In other compatible embodiments, DNA encoding the desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which may be modified as described herein) may be used to clone constant and variable region sequences for the manufacture antibodies as described in Newman et al., U.S. Pat. No. 5,658,570 which is incorporated by reference herein. Essentially, this entails extraction of RNA from the selected cells, conversion to cDNA, and amplification thereof by PCR using Ig specific primers. As will be discussed in more detail below, transformed cells expressing the desired antibody may be grown up in relatively large quantities to provide clinical and commercial supplies of the immunoglobulin.

Those skilled in the art will also appreciate that DNA encoding antibodies or antibody fragments may also be derived from antibody phage libraries as set forth, for example, in EP 368 684 B1 and U.S. Pat. No. 5,969,108 each of which is incorporated herein by reference. Several publications (e.g., Marks et al. Bio/Technology 10:779-783 (1992)) have described the production of high affinity human antibodies by chain shuffling, as well as combinatorial infection and in vivo recombination as a strategy for constructing large phage libraries. Such procedures provide viable alternatives to traditional hybridoma techniques for the isolation and subsequent cloning of monoclonal antibodies and, as such, are clearly within the purview of the instant invention.

Yet other embodiments of the present invention comprise the generation of substantially human antibodies in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array in such germ line mutant mice will result in the production of human antibodies upon antigen challenge. Another preferred means of generating human antibodies using SCID mice is disclosed in commonly-owned, co-pending U.S. Pat. No. 5,811,524 which is incorporated herein by reference. It will be appreciated that the genetic material associated with these human antibodies may also be isolated and manipulated as described herein.

Yet another highly efficient means for generating recombinant antibodies is disclosed by Newman, Biotechnology, 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

As is apparent from the instant specification, genetic sequences useful for producing the modified antibodies of the present invention may be obtained from a number of different sources. For example, as discussed extensively above, a variety of human antibody genes are available in the form of publicly accessible deposits. Many sequences of antibodies and antibody-encoding genes have been published and suitable antibody genes can be synthesized from these sequences much as previously described. Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

It will further be appreciated that the scope of this invention encompasses all alleles, variants and mutations of the DNA sequences described herein.

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligodT cellulose. Techniques suitable to these purposes are familiar in the art and are described in the foregoing references.

cDNAs that encode the light and the heavy chains of the antibody may be made, either simultaneously or separately, using reverse transcriptase and DNA polymerase in accordance with well known methods. It may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes.

DNA, typically plasmid DNA, may be isolated from the cells as described herein, restriction mapped and sequenced in accordance with standard, well known techniques set forth in detail in the foregoing references relating to recombinant DNA techniques. Of course, the DNA may be modified according to the present invention at any point during the isolation process or subsequent analysis.

Preferred antibody sequences are disclosed herein. Oligonucleotide synthesis techniques compatible with this aspect of the invention are well known to the skilled artisan and may be carried out using any of several commercially available automated synthesizers. In addition, DNA sequences encoding several types of heavy and light chains set forth herein can be obtained through the services of commercial DNA synthesis vendors. The genetic material obtained using any of the foregoing methods may then be altered or modified to provide antibodies compatible with the present invention.

While a variety of different types of antibodies may be obtained and modified according to the instant invention, the modified antibodies of the instant invention will share various common traits. To that end, the term “immunoglobulin” shall be held to refer to a tetramer (2 heavy and 2 light chains) or aggregate thereof whether or not it possesses any relevant specific immunoreactivity. “Antibodies” refers to such assemblies which have significant known specific immunoreactive activity to an antigen (e.g. a tumor associated antigen), comprising light and heavy chains, with or without covalent linkage between them. As discussed above, “modified antibodies” according to the present invention are held to mean antibodies, or immunoreactive fragments or recombinants thereof, in which at least a fraction of one or more of the constant region domains has been deleted or otherwise altered so as to provide desired biochemical characteristics such as increased tumor localization or reduced serum half-life when compared with a whole, unaltered antibody of approximately the same immunogenicity. For the purposes of the instant application, immunoreactive single chain antibody constructs having altered or omitted constant region domains may be considered to be modified antibodies. As discussed above, preferred modified antibodies of the present invention have at least a portion of one of the constant domains deleted. More preferably, one entire domain of the constant region of the modified antibody will be deleted and even more preferably the entire C_{H}2 domain will be deleted.

Basic immunoglobulin structures in vertebrate systems are relatively well understood. As will be discussed in more detail below, the generic term “immunoglobulin” comprises five distinct classes of antibody that can be distinguished biochemically. While all five classes are clearly within the scope of the present invention, the following discussion will generally be directed to the class of IgG molecules. With regard to IgG, immunoglobulins comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

More specifically, both the light and heavy chains are divided into regions of structural and functional homology. The terms “constant” and “variable” are used functionally. In this regard, it will be appreciated that the variable domains of both the light (V_{L}) and heavy (V_{H}) chains determine antigen recognition and specificity. Conversely, the constant domains of the light chain (C_{L}) and the heavy chain (C_{H}1, C_{H}2 or C_{H}3) confer important biological properties such as secretion, transplacental mobility, Fc receptor binding, complement binding, and the like. By convention the numbering of the constant region domains increases as they become more distal from the antigen binding site or amino-terminus of the antibody. Thus, the C_{H}3 and C_{L} domains actually comprise the carboxy-terminus of the heavy and light chains respectively.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages when the immunogobulins are generated either by hybridomas, B cells or genetically engineered host cells. However, if non-covalent association of the chains can be effected in the correct geometry, the aggregate of non-disulfide-linked chains will still be capable of reaction with antigen. In the heavy chain, the amino acid sequences run from an N-terminus at the forked ends of the Y configuration to the C-terminus at the bottom of each chain. At the N-terminus is a variable region and at the C-terminus is a constant region. Those skilled in the art will appreciate that heavy chains are classified as gamma, mu, alpha, delta, or epsilon, (γ, μ, α, δ, ε) with some subclasses among them. It is the nature of this chain that determines the “class” of the antibody as IgA, IgD, IgE IgG, or IgM. The immunoglobulin subclasses (isotypes) e.g. IgG₁, IgG₂, IgG₃, IgG₄, IgA₁, etc. are well characterized and are known to confer functional specialization. Modified versions of each of these classes and isotypes are readily discernable to the skilled artisan in view of the instant disclosure and, accordingly, are within the purview of the instant invention.

As indicated above, the variable region allows the antibody to selectively recognize and specifically bind epitopes on immunoreactive antigens. That is, the V_{L} domain and V_{H} domain of an antibody combine to form the variable region that defines a three dimensional antigen binding site. This quaternary antibody structure provides for an antigen binding site present at the end of each arm of the Y. More specifically, the antigen binding site is defined by three complementary determining regions (CDRs) on each of the V_{H} and V_{L} chains.

The six CDRs are short, non-contiguous sequences of amino acids that are specifically positioned to form the antigen binding site as the antibody assumes its three dimensional configuration in an aqueous environment. The remainder of the heavy and light variable domains show less inter-molecular variability in amino acid sequence and are termed the framework regions. The framework regions largely adopt a β-sheet conformation and the CDRs form loops connecting, and in some cases forming part of, the β-sheet structure. Thus, these framework regions act to form a scaffold that provides for positioning the six CDRs in correct orientation by inter-chain, non-covalent interactions. In any event, the antigen binding site formed by the positioned CDRs defines a surface complementary to the epitope on the immunoreactive antigen. This complementary surface promotes the non-covalent binding of the antibody to the immunoreactive antigen epitope.

For the purposes of the present invention, it should be appreciated that the disclosed modified antibodies may comprise any type of variable region that provides for the association of the antibody with the selected tumor associated antigen. In this regard, the variable region may comprise or be derived from any type of mammal that can be induced to mount a humoral response and generate immunoglobulins against the desired tumor associated antigen. As such, the variable region of the modified antibodies may be, for example, of human, murine, non-human primate (e.g. cynomolgus monkeys, macaques, etc.) or lupine origin. In particularly preferred embodiments both the variable and constant regions of the modified immunoglobulins are human. In other selected embodiments the variable regions of compatible antibodies (usually derived from a non-human source) may be engineered or specifically tailored to improve the binding properties or reduce the immunogenicity of the molecule. In this respect, variable regions useful in the present invention may be humanized or otherwise altered through the inclusion of imported amino acid sequences.

By “humanized antibody” is meant an antibody derived from a non-human source, typically a murine antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81: 6851-5 (1984); Morrison et al., Adv. Immunol. 44: 65-92 (1988); Verhoeyen et al., Science 239: 1534-1536 (1988); Padlan, Molec. Immun. 28: 489-498 (1991); Padlan, Molec. Immun. 31: 169-217 (1994), and U.S. Pat. Nos. 5,585,089, 5,693,761 and 5,693,762 all of which are hereby incorporated by reference in their entirety.

Those skilled in the art will appreciate that the technique set forth in option (a) above will produce “classic” chimeric antibodies. In the context of the present application the term “chimeric antibodies” will be held to mean any antibody wherein the immunoreactive region or site is obtained or derived from a first species and the constant region (which may be intact, partial or modified in accordance with the instant invention) is obtained from a second species. In preferred embodiments the antigen binding region or site will be from a non-human source (e.g. mouse) and the constant region is human. While the immunogenic specificity of the variable region is not generally affected by its source, a human constant region is less likely to elicit an immune response from a human subject than would the constant region from a non-human source.

Preferably, the variable domains in both the heavy and light chains are altered by at least partial replacement of one or more CDRs and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. It must be emphasized that it may not be necessary to replace all of the CDRs with the complete CDRs from the donor variable region to transfer the antigen binding capacity of one variable domain to another. Rather, it may only be necessary to transfer those residues that are necessary to maintain the activity of the antigen binding site. Given the explanations set forth in U.S. Pat. Nos. 5,585,089, 5,693,761 and 5,693,762, it will be well within the competence of those skilled in the art, either by carrying out routine experimentation or by trial and error testing to obtain a functional antibody with reduced immunogenicity.

Alterations to the variable region notwithstanding, those skilled in the art will appreciate that the modified antibodies of the instant invention will comprise antibodies, or immunoreactive fragments thereof, in which at least a fraction of one or more of the constant region domains has been deleted or otherwise altered so as to provide desired biochemical characteristics such as increased tumor localization or reduced serum half-life when compared with an antibody of approximately the same immunogenicity comprising a native or unaltered constant region. In preferred embodiments, the constant region of the modified antibodies will comprise a human constant region. Modifications to the constant region compatible with the instant invention comprise additions, deletions or substitutions of one or more amino acids in one or more domains. That is, the modified antibodies disclosed herein may comprise alterations or modfications to one or more of the three heavy chain constant domains (C_{H}1, C_{H}2 or C_{H}3) and/or to the light chain constant domain (C_{L}). As will be discussed in more detail below and shown in the examples, preferred embodiments of the invention comprise modified constant regions wherein one or more domains are partially or entirely deleted. In especially preferred embodiments the modified antibodies will comprise domain deleted constructs or variants wherein the entire C_{H}2 domain has been removed (ΔC_{H}2 constructs). In still other preferred embodiments the omitted constant region domain will be replaced by a short amino acid spacer (e.g. 10 residues) that provides some of the molecular flexibility typically imparted by the absent constant region.

As previously indicated, the subunit structures and three dimensional configuration of the constant regions of the various immunoglobulin classes are well known. For example, the C_{H}2 domain of a human IgG Fc region usually extends from about residue 231 to residue 340 using conventional numbering schemes. The C_{H}2 domain is unique in that it is not closely paired with another domain. Rather, two N-linked branched carbohydrate chains are interposed between the two C_{H}2 domains of an intact native IgG molecule. It is also well documented that the C_{H}3 domain extends from the C_{H}2 domain to the C-terminal of the IgG molecule and comprises approximately 108 residues while the hinge region of an IgG molecule joins the C_{H}2 domain with the C_{H}1 domain. This hinge region encompasses on the order of 25 residues and is flexible, thereby allowing the two N-terminal antigen binding regions to move independently.

Besides their configuration, it is known in the art that the constant region mediates several effector functions. For example, binding of the C1 component of complement to antibodies activates the complement system. Activation of complement is important in the opsonisation and lysis of cell pathogens. The activation of complement also stimulates the inflammatory response and may also be involved in autoimmune hypersensitivity. Further, antibodies bind to cells via the Fc region, with a Fc receptor site on the antibody Fc region binding to a Fc receptor (FcR) on a cell. There are a number of Fc receptors which are specific for different classes of antibody, including IgG (gamma receptors), IgE (eta receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. Although various Fc receptors and receptor sites have been studied to a certain extent, there is still much which is unknown about their location, structure and functioning.

While not limiting the scope of the present invention, it is believed that antibodies comprising constant regions modified as described herein provide for altered effector functions that, in turn, affect the biological profile of the administered antibody. For example, the deletion or inactivation (through point mutations or other means) of a constant region domain may reduce Fc receptor binding of the circulating modified antibody thereby increasing tumor localization. In other cases it may be that constant region modifications consistent with the instant invention moderate compliment binding and thus reduce the serum half life and nonspecific association of a conjugated cytotoxin. Yet other modifications of the constant region may be used to eliminate disulfide linkages or oligosaccharide moities that allow for enhanced localization due to increased antigen specificity or antibody flexibility. More generally, those skilled in the art will realize that antibodies modified as described herein may exert a number of subtle effects that may or may not be appreciated. However, as shown in the examples below, the resulting physiological profile, bioavailability and other biochemical effects of the modifications, such as tumor localization and serum half-life, may easily be measured and quantified using well known immunology techniques without undue experimentation.

Similarly, modifications to the constant region in accordance with the instant invention may easily be made using well known biochemical or molecular engineering techniques well within the purview of the skilled artisan. In this respect the examples appended hereto provide various constructs having constant regions modified in accordance with the present invention. More specifically, the exemplified constructs comprise chimeric and humanized antibodies having human constant regions that have been engineered to delete the C_{H}2 domain. Those skilled in the art will appreciate that such constructs are particularly preferred due to the regulatory properties of the C_{H}2 domain on the catabolic rate of the antibody.

The ΔC_{H}2 domain deleted antibodies set forth in the examples and the Figures are derived from chimeric C2B8 antibody which is immunospecific for the CD20 pan B cell antigen and humanized CC49 antibody which is specific for the TAG 72 antigen. As discussed in more detail below, both domain deleted constructs were derived from a proprietary vector (IDEC Pharmaceuticals, San Diego) encoding an IgG1 human constant domain. Essentially, the vector was engineered to delete the C_{H}2 domain and provide a modified vector expressing a domain deleted IgG1 constant region. Genes encoding the murine variable region of the C2B8 antibody or the variable region of the humanized CC49 antibody were then inserted in the modified vector and cloned. When expressed in transformed cells, these vectors provided huCC49.ΔC_{H}2 or C2B8.ΔC_{H}2 respectively. As illustrated herein, these constructs exhibited a number of properties that make them particularly attractive candidates for use in myelosuppresed cancer patients or in cancer patients that are undergoing potentially myelosuppressive adjunct treatments.

It will be noted that the foregoing exemplary constructs were engineered to fuse the C_{H}3 domain directly to the hinge region of the respective modified antibodies. In other constructs it may be desirable to provide a peptide spacer between the hinge region and the modified C_{H}2 and/or C_{H}3 domains. For example, compatible constructs could be expressed wherein the C_{H}2 domain has been deleted and the remaining C_{H}3 domain (modified or unmodified) is joined to the hinge region with a 5-20 amino acid spacer. In this respect, one preferred spacer has the amino acid sequence IGKTISKKAK (Seq. ID No. 1). Such a spacer may be added, for instance, to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, it is preferable that any spacer added to the construct be relatively non-immunogenic or, even more preferably, omitted altogether if the desired biochemical qualities of the modified antibodies may be maintained.

Besides the deletion of whole constant region domains, it will be appreciated that the antibodies of the present invention may be provided by the partial deletion or substitution of a few or even a single amino acid. For example, the mutation of a single amino acid in selected areas of the C_{H}2 domain may be enough to substantially reduce Fc binding and thereby increase tumor localization. Similarly, it may be desirable to simply delete that part of one or more constant region domains that control the effector function (e.g. complement CLQ binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g. Fc binding) while substantially maintaining the configuration and immunogenic profile of the modified antibody. Yet other preferred embodiments may comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

Following manipulation of the isolated genetic material to provide modified antibodies as set forth above, the genes are typically inserted in an expression vector for introduction into host cells that may be used to produce the desired quantity of modified antibody.

The term “vector” or “expression vector” is used herein for the purposes of the specification and claims, to mean vectors used in accordance with the present invention as a vehicle for introducing into and expressing a desired gene in a cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

In particularly preferred embodiments the cloned variable region genes are inserted into an expression vector along with the heavy and light chain constant region genes (preferably human) modified as discussed above. Preferably, this is effected using a proprietary expression vector of IDEC, Inc., referred to as NEOSPLA. This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. As seen in the examples below, this vector has been found to result in very high level expression of antibodies upon incorporation of variable and constant region genes, transfection in CHO cells, followed by selection in G418 containing medium and methotrexate amplification. This vector system is substantially disclosed in commonly assigned U.S. Pat. Nos. 5,736,137 and 5,658,570, each of which is incorporated by reference in its entirety herein. This system provides for high expression levels, i.e., >30 pg/cell/day.

In other preferred embodiments the modified antibodies of the instant invention may be expressed using polycistronic constructs such as those disclosed in copending U.S. provisional application No. 60/331,481 filed Nov. 16, 2001 and incorporated herein in its entirety. In these novel expression systems, multiple gene products of interest such as heavy and light chains of antibodies may be produced from a single polycistronic construct. These systems advantageously use an internal ribosome entry site (IRES) to provide relatively high levels of modified antibodies in eukaryotic host cells. Compatible IRES sequences are disclosed in U.S. Pat. No. 6,193,980 which is also incorporated herein. Those skilled in the art will appreciate that such expression systems may be used to effectively produce the full range of modified antibodies disclosed in the instant application.

More generally, once the vector or DNA sequence containing the modified antibody has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or flourescence-activated cell sorter analysis (FACS), immunohistochemistry and the like.

As used herein, the term “transformation” shall be used in a broad sense to refer to any introduction of DNA into a recipient host cell that changes the genotype and consequently results in a change in the recipient cell.

Along those same lines, “host cells” refers to cells that have been transformed with vectors constructed using recombinant DNA techniques and containing at least one heterologous gene. As defined herein, the antibody or modification thereof produced by a host cell is by virtue of this transformation. In descriptions of processes for isolation of antibodies from recombinant hosts, the terms “cell” and “cell culture” are used interchangeably to denote the source of antibody unless it is clearly specified otherwise. In other words, recovery of antibody from the “cells” may mean either from spun down whole cells, or from the cell culture containing both the medium and the suspended cells.

The host cell line used for protein expression is most preferably of mammalian origin; those skilled in the art are credited with ability to preferentially determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3.times.63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature.

In vitro production allows scale-up to give large amounts of the desired antibodies. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. For isolation of the modified antibodies, the immunoglobulins in the culture supernatants are first concentrated, e.g. by precipitation with ammonium sulphate, dialysis against hygroscopic material such as PEG, filtration through selective membranes, or the like. If necessary and/or desired, the concentrated antibodies are purified by the customary chromatography methods, for example gel filtration, ion-exchange chromatography, chromatography over DEAE-cellulose or (immuno-)affinity chromatography.

The modified immunoglobulin genes can also be expressed non-mammalian cells such as bacteria or yeast. In this regard it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli; Salmonella; Bacillaceae, such as Bacillus subtilis; Pneumococcus; Streptococcus, and Haemophilus influenzae. It will further be appreciated that, when expressed in bacteria, the immunoglobulin heavy chains and light chains typically become part of inclusion bodies. The chains then must be isolated, purified and then assembled into functional immunoglobulin molecules.

In addition to prokaryates, eukaryotic microbes may also be used. Saccharomyces cerevisiae, or common baker's yeast, is the most commonly used among eukaryotic microorganisms although a number of other strains are commonly available. For expression in Saccharomyces, the plasmid YRp7, for example, (Stinchcomb et al., Nature, 282:39 (1979); Kingsman et al., Gene, 7:141 (1979); Tschemper et al., Gene, 10:157 (1980)) is commonly used. This plasmid already contains the trpl gene which provides a selection marker for a mutant strain of yeast lacking the ability to grow in tryptophan, for example ATCC No. 44076 or PEP4-1 (Jones, Genetics, 85:12 (1977)). The presence of the trpl lesion as a characteristic of the yeast host cell genome then provides an effective environment for detecting transformation by growth in the absence of tryptophan.

Regardless of how clinically useful quantities are obtained, the modified antibodies of the present invention may be used in any one of a number of conjugated (i.e. an immunoconjugate) or unconjugated forms. In particular, the antibodies of the present invention may be conjugated to cytotoxins such as radioisotopes, therapeutic agents, cytostatic agents, biological toxins or prodrugs. Alternatively, the modified antibodies of the instant invention may be used in a nonconjugated or “naked” form to harness the subject's natural defense mechanisms including complement-dependent cytotoxicity (CDC) and antibody dependent cellular toxicity (ADCC) to eliminate the malignant cells. In particularly preferred embodiments, the modified antibodies may be conjugated to radioisotopes, such as ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re using anyone of a number of well known chelators or direct labeling. In other embodiments, the disclosed compositions may comprise modified antibodies coupled to drugs, prodrugs or biological response modifiers such as methotrexate, adriamycin, and lymphokines such as interferon. Still other embodiments of the present invention comprise the use of modified antibodies conjugated to specific biotoxins such as ricin or diptheria toxin. In yet other embodiments the modified antibodies may be complexed with other immunologically active ligands (e.g. antibodies or fragments thereof) wherein the resulting molecule binds to both the neoplastic cell and an effector cell such as a T cell. The selection of which conjugated or unconjugated modified antibody to use will depend of the type and stage of cancer, use of adjunct treatment (e.g., chemotherapy or external radiation) and patient condition. It will be appreciated that one skilled in the art could readily make such a selection in view of the teachings herein.

As used herein, “a cytotoxin or cytotoxic agent” means any agent that is detrimental to the growth and proliferation of cells and may act to reduce, inhibit or distroy a malignancy when exposed thereto. Exemplary cytotoxins include, but are not limited to, radionuclides, biotoxins, cytostatic or cytotoxic therapeutic agents, prodrugs, immunologically active ligands and biological response modifiers such as cytokines As will be discussed in more detail below, radionuclide cytotoxins are particularly preferred for use in the instant invention. However, any cytotoxin that acts to retard or slow the growth of malignant cells or to eliminate malignant cells and may be associated with the modified antibodies disclosed herein is within the purview of the present invention.

It will be appreciated that, in previous studies, anti-tumor antibodies labeled with isotopes have been used successfully to destroy cells in solid tumors as well as lymphomas/leukemias in animal models, and in some cases in humans. The radionuclides act by producing ionizing radiation which causes multiple strand breaks in nuclear DNA, leading to cell death. The isotopes used to produce therapeutic conjugates typically produce high energy α-, γ- or β-particles which have a therapeutically effective path length. Such radionuclides kill cells to which they are in close proximity, for example neoplastic cells to which the conjugate has attached or has entered. They generally have little or no effect on non-localized cells. Radionuclides are essentially non-immunogenic.

With respect to the use of radiolabeled conjugates in conjunction with the present invention, the modified antibodies may be directly labeled (such as through iodination) or may be labeled indirectly through the use of a chelating agent. As used herein, the phrases “indirect labeling” and “indirect labeling approach” both mean that a chelating agent is covalently attached to an antibody and at least one radionuclide is associated with the chelating agent. Such chelating agents are typically referred to as bifunctional chelating agents as they bind both the polypeptide and the radioisotope. Particularly preferred chelating agents comprise 1-isothiocycmatobenzyl-3-methyldiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Particularly preferred radionuclides for indirect labeling include ¹¹¹In and ⁹⁰Y.

As used herein, the phrases “direct labeling” and “direct labeling approach” both mean that a radionuclide is covalently attached directly to an antibody (typically via an amino acid residue). More specifically, these linking technologies include random labeling and site-directed labeling. In the latter case, the labeling is directed at specific sites on the dimer or tetramer, such as the N-linked sugar residues present only on the Fc portion of the conjugates. Further, various direct labeling techniques and protocols are compatible with the instant invention. For example, Technetium-99m labelled antibodies may be prepared by ligand exchange processes, by reducing pertechnate (TcO₄⁻) with stannous ion solution, chelating the reduced technetium onto a Sephadex column and applying the antibodies to this column, or by batch labelling techniques, e.g. by incubating pertechnate, a reducing agent such as SnCl₂, a buffer solution such as a sodium-potassium phthalate-solution, and the antibodies. In any event, preferred radionuclides for directly labeling antibodies are well known in the art and a particularly preferred radionuclide for direct labeling is ¹³¹I covalently attached via tyrosine residues. Modified antibodies according to the invention may be derived, for example, with radioactive sodium or potassium iodide and a chemical oxidising agent, such as sodium hypochlorite, chloramine T or the like, or an enzymatic oxidising agent, such as lactoperoxidase, glucose oxidase and glucose. However, for the purposes of the present invention, the indirect labeling approach is particularly preferred.

Patents relating to chelators and chelator conjugates are known in the art. For instance, U.S. Pat. No. 4,831,175 of Gansow is directed to polysubstituted diethylenetriaminepentaacetic acid chelates and protein conjugates containing the same, and methods for their preparation. U.S. Pat. Nos. 5,099,069, 5,246,692, 5,286,850, 5,434,287 and 5,124,471 of Gansow also relate to polysubstituted DTPA chelates. These patents are incorporated herein in their entirety. Other examples of compatible metal chelators are ethylenediaminetetraacetic acid (EDTA), diethylenetriaminepentaacetic acid (DPTA), 1,4,8,11-tetraazatetradecane, 1,4,8,11-tetraazatetradecane-1,4,8,11-tetraacetic acid, 1-oxa-4,7,12,15-tetraazaheptadecane-4,7,12,15-tetraacetic acid, or the like. Cyclohexyl-DTPA or CHX-DTPA is particularly preferred and is exemplified extensively below. Still other compatible chelators, including those yet to be discovered, may easily be discerned by a skilled artisan and are clearly within the scope of the present invention.

Compatible chelators, including the specific bifunctional chelator used to facilitate chelation in co-pending application Ser. Nos. 08/475,813, 08/475,815 and 08/478,967, are preferably selected to provide high affinity for trivalent metals, exhibit increased tumor-to-non-tumor ratios and decreased bone uptake as well as greater in vivo retention of radionuclide at target sites, i.e., B-cell lymphoma tumor sites. However, other bifunctional chelators that may or may not possess all of these characteristics are known in the art and may also be beneficial in tumor therapy.

It will also be appreciated that, in accordance with the teachings herein, modified antibodies may be conjugated to different radiolabels for diagnostic and therapeutic purposes. To this end the aforementioned co-pending applications, herein incorporated by reference in their entirety, disclose radiolabeled therapeutic conjugates for diagnostic “imaging” of tumors before administration of therapeutic antibody. “In2B8” conjugate comprises a murine monoclonal antibody, 2B8, specific to human CD20 antigen, that is attached to ¹¹¹In via a bifunctional chelator, i.e., MX-DTPA (diethylenetriaminepentaacetic acid), which comprises a 1:1 mixture of 1-isothiocyanatobenzyl-3-methyl-DTPA and 1-methyl-3-isothiocyanatobenzyl-DTPA. ¹¹¹In is particularly preferred as a diagnostic radionuclide because between about 1 to about 10 mCi can be safely administered without detectable toxicity; and the imaging data is generally predictive of subsequent ⁹⁰Y-labeled antibody distribution. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, J. Nuc. Med. 26: 3328 (1985) and Carraguillo et al., J. Nuc. Med. 26: 67 (1985).

As indicated above, a variety of radionuclides are applicable to the present invention and those skilled in the art are credited with the ability to readily determine which radionuclide is most appropriate under various circumstances. For example, ¹³¹I is a well known radionuclide used for targeted immunotherapy. However, the clinical usefulness of ¹³¹I can be limited by several factors including: eight-day physical half-life; dehalogenation of iodinated antibody both in the blood and at tumor sites; and emission characteristics (e.g., large gamma component) which can be suboptimal for localized dose deposition in tumor. With the advent of superior chelating agents, the opportunity for attaching metal chelating groups to proteins has increased the opportunities to utilize other radionuclides such as ¹¹¹In and ⁹⁰Y. ⁹⁰Y provides several benefits for utilization in radioimmunotherapeutic applications: the 64 hour half-life of ⁹⁰Y is long enough to allow antibody accumulation by tumor and, unlike e.g., ¹³¹I, ⁹⁰Y is a pure beta emitter of high energy with no accompanying gamma irradiation in its decay, with a range in tissue of 100 to 1,000 cell diameters. Furthermore, the minimal amount of penetrating radiation allows for outpatient administration of ⁹⁰Y-labeled antibodies. Additionally, internalization of labeled antibody is not required for cell killing, and the local emission of ionizing radiation should be lethal for adjacent tumor cells lacking the target antigen.

Effective single treatment dosages (i.e., therapeutically effective amounts) of ⁹⁰Y-labeled modified antibodies range from between about 5 and about 75 mCi, more preferably between about 10 and about 40 mCi. Effective single treatment non-marrow ablative dosages of ¹³¹I-labeled antibodies range from between about 5 and about 70 mCi, more preferably between about 5 and about 40 mCi. Effective single treatment ablative dosages (i.e., may require autologous bone marrow transplantation) of ¹³¹I-labeled antibodies range from between about 30 and about 600 mCi, more preferably between about 50 and less than about 500 mCi. In conjunction with a chimeric antibody, owing to the longer circulating half life vis-á-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the ¹¹¹In label, are typically less than about 5 mCi.

While a great deal of clinical experience has been gained with ¹³¹I and ⁹⁰Y, other radiolabels are known in the art and have been used for similar purposes. Still other radioisotopes are used for imaging. For example, additional radioisotopes which are compatible with the scope of the instant invention include, but are not limited to, ¹²³I, ¹²⁵I, ³²P, ⁵⁷Co, ⁶⁴Cu, ⁶⁷Cu, ⁷⁷Br, ⁸¹Rb, ⁸¹Kr, ⁸⁷Sr, ¹¹³In, ¹²⁷Cs, ¹²⁹Cs, ¹³²I, ¹⁹⁷Hg, ²⁰³Pb, ²⁰⁶Bi, ¹⁷⁷Lu, ¹⁸⁶Re, ²¹²Pb, ²¹²Bi, ⁴⁷Sc, ¹⁰⁵Rh, ¹⁰⁹Pd, ¹⁵³Sm, ¹⁸⁸Re, ¹⁹⁹Au, ²²⁵Ac, ²¹¹At, and ²¹³Bi. In this respect alpha, gamma and beta emitters are all compatible with in the instant invention. Further, in view of the instant disclosure it is submitted that one skilled in the art could readily determine which radionuclides are compatible with a selected course of treatment without undue experimentation. To this end, additional radionuclides which have already been used in clinical diagnosis include ¹²⁵I, ¹²³I, ⁹⁹Tc, ⁴³K, ⁵²Fe, ⁶⁷Ga, ⁶⁸Ga, as well as ¹¹¹In. Antibodies have also been labeled with a variety of radionuclides for potential use in targeted immunotherapy Peirersz et al. Immunol. Cell Biol. 65: 111-125 (1987). These radionuclides include ¹⁸⁸Re and ¹⁸⁶Re as well as ¹⁹⁹Au and ⁶⁷Cu to a lesser extent. U.S. Pat. No. 5,460,785 provides additional data regarding such radioisotopes and is incorporated herein by reference.

In addition to radionuclides, the modified antibodies of the present invention may be conjugated to, or associated with, any one of a number of biological response modifiers, pharmaceutical agents, toxins or immunologically active ligands. Those skilled in the art will appreciate that these non-radioactive conjugates may be assembled using a variety of techniques depending on the selected cytotoxin. For example, conjugates with biotin are prepared e.g. by reacting the modified antibodies with an activated ester of biotin such as the biotin N-hydroxysuccinimide ester. Similarly, conjugates with a fluorescent marker may be prepared in the presence of a coupling agent, e.g. those listed above, or by reaction with an isothiocyanate, preferably fluorescein-isothiocyanate. Conjugates of the chimeric antibodies of the invention with cytostatic/cytotoxic substances and metal chelates are prepared in an analogous manner.

Preferred agents for use in the present invention are cytotoxic drugs, particularly those which are used for cancer therapy. Such drugs include, in general, cytostatic agents, alkylating agents, antimetabolites, anti-proliferative agents, tubulin binding agents, hormones and hormone antagonists, and the like. Exemplary cytostatics that are compatible with the present invention include alkylating substances, such as mechlorethamine, triethylenephosphoramide, cyclophosphamide, ifosfamide, chlorambucil, busulfan, melphalan or triaziquone, also nitrosourea compounds, such as carmustine, lomustine, or semustine. Other preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, carminomycin, daunorubicin (daunomycin), doxorubicin, aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, or podophyllotoxin derivatives such as etoposide or etoposide phosphate, melphalan, vinblastine, vincristine, leurosidine, vindesine, leurosine and the like. Still other cytotoxins that are compatible with the teachings herein include taxol, taxane, cytochalasin B, gramicidin D, ethidium bromide, emetine, tenoposide, colchicin, dihydroxy anthracin dione, mitoxantrone, procaine, tetracaine, lidocaine, propranolol, and puromycin and analogs or homologs thereof. Hormones and hormone antagonists, such as corticosteroids, e.g. prednisone, progestins, e.g. hydroxyprogesterone or medroprogesterone, estrogens, e.g. diethylstilbestrol, antiestrogens, e.g. tamoxifen, androgens, e.g. testosterone, and aromatase inhibitors, e.g. aminogluthetimide are also compatible with the teachings herein. As noted previously, one skilled in the art may make chemical modifications to the desired compound in order to make reactions of that compound more convenient for purposes of preparing conjugates of the invention.

One example of particularly preferred cytotoxins comprise members or derivatives of the enediyne family of anti-tumor antibiotics, including calicheamicin, esperamicins or dynemicins. These toxins are extremely potent and act by cleaving nuclear DNA, leading to cell death. Unlike protein toxins which can be cleaved in vivo to give many inactive but immunogenic polypeptide fragments, toxins such as calicheamicin, esperamicins and other enediynes are small molecules which are essentially non-immunogenic. These non-peptide toxins are chemically-linked to the dimers or tetramers by techniques which have been previously used to label monoclonal antibodies and other molecules. These linking technologies include site-specific linkage via the N-linked sugar residues present only on the Fc portion of the conjugates. Such site-directed linking methods have the advantage of reducing the possible effects of linkage on the binding properties of the conjugate.

As previously alluded to, compatible cytotoxins may comprise a prodrug. As used herein, the term “prodrug” refers to a precursor or derivative form of a pharmaceutically active substance that is less cytotoxic to tumor cells compared to the parent drug and is capable of being enzymatically activated or converted into the more active parent form. Prodrugs compatible with the invention include, but are not limited to, phosphate-containing prodrugs, thiophosphate-containing prodrugs, sulfate containing prodrugs, peptide containing prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. Further examples of cytotoxic drugs that can be derivatized into a prodrug form for use in the present invention comprise those chemotherapeutic agents described above.

Among other cytotoxins, it will be appreciated that the antibody can also be associated with a biotoxin such as ricin subunit A, abrin, diptheria toxin, botulinum, cyanginosins, saxitoxin, shigatoxin, tetanus, tetrodotoxin, trichothecene, verrucologen or a toxic enzyme. Preferably, such constructs will be made using genetic engineering techniques that allow for direct expression of the antibody-toxin construct. Other biological response modifiers that may be associated with the modified antibodies of the present invention comprise cytokines such as lymphokines and interferons. Moreover, as indicated above, similar constructs may also be used to associate immunologically active ligands (e.g. antibodies or fragments thereof) with the modified antibodies of the present invention. Preferably, these immunologically active ligands would be directed to antigens on the surface of immunoactive effector cells. In these cases, the constructs could be used to bring effector cells, such as T cells or NK cells, in close proximity to the neoplastic cells bearing a tumor associated antigen thereby provoking the desired immune response. In view of the instant disclosure it is submitted that one skilled in the art could readily form such constructs using conventional techniques.

Another class of compatible cytotoxins that may be used in conjunction with the disclosed modified antibodies are radiosensitizing drugs that may be effectively directed to tumor cells. Such drugs enhance the sensitivity to ionizing radiation, thereby increasing the efficacy of radiotherapy. An antibody conjugate internalized by the tumor cell would deliver the radiosensitizer nearer the nucleus where radiosensitization would be maximal. The unbound radiosensitizer linked modified antibodies would be cleared quickly from the blood, localizing the remaining radiosensitization agent in the target tumor and providing minimal uptake in normal tissues. After rapid clearance from the blood, adjunct radiotherapy would be administered in one of three ways: 1.) external beam radiation directed specifically to the tumor, 2.) radioactivity directly implanted in the tumor or 3.) systemic radioimmunotherapy with the same targeting antibody. A potentially attractive variation of this approach would be the attachment of a therapeutic radioisotope to the radiosensitized immuno conjugate, thereby providing the convenience of administering to the patient a single drug.

Whether or not the disclosed antibodies are used in a conjugated or unconjugated form, it will be appreciated that a major advantage of the present invention is the ability to use these antibodies in myelosuppressed patients, especially those who are undergoing, or have undergone, adjunct therapies such as radiotherapy or chemotherapy. That is, the beneficial delivery profile (i.e. relatively short serum dwell time and enhanced localization) of the modified antibodies makes them particularly useful for treating patients that have reduced red marrow reserves and are sensitive to myelotoxicity. In this regard, the unique delivery profile of the modified antibodies make them very effective for the administration of radiolabeled conjugates to myelosuppressed cancer patients. As such, the modified antibodies are useful in a conjugated or unconjugated form in patients that have previously undergone adjunct therapies such as external beam radiation or chemotherapy. In other preferred embodiments, the modified antibodies (again in a conjugated or unconjugated form) may be used in a combined therapeutic regimen with chemotherapeutic agents. Those skilled in the art will appreciate that such therapeutic regimens may comprise the sequential, simultaneous, concurrent or coextensive administration of the disclosed antibodies and one or more chemotherapeutic agents. Particularly preferred embodiments of this aspect of the invention will comprise the administration of a radiolabeled antibody.

While the modified antibodies may be administered as described immediately above, it must be emphasized that in other embodiments conjugated and unconjugated modified antibodies may be administered to otherwise healthy cancer patients as a first line therapeutic agent. In such embodiments the modified antibodies may be administered to patients having normal or average red marrow reserves and/or to patients that have not, and are not, undergoing adjunct therapies such as external beam radiation or chemotherapy.

However, as discussed above, selected embodiments of the invention comprise the administration of modified antibodies to myelosuppressed patients or in combination or conjunction with one or more adjunct therapies such as radiotherapy or chemotherapy (i.e. a combined therapeutic regimen). As used herein, the administration of modified antibodies in conjunction or combination with an adjunct therapy means the sequential, simultaneous, coextensive, concurrent, concomitant or contemporaneous administration or application of the therapy and the disclosed antibodies. Those skilled in the art will appreciate that the administration or application of the various components of the combined therapeutic regimen may be timed to enhance the overall effectiveness of the treatment. For example, chemotherapeutic agents could be administered in standard, well known courses of treatment followed within a few weeks by radioimmunoconjugates of the present invention. Conversely, cytotoxin associated modified antibodies could be administered intravenously followed by tumor localized external beam radiation. In yet other embodiments, the modified antibody may be administered concurrently with one or more selected chemotherapeutic agents in a single office visit. A skilled artisan (e.g. an experienced oncologist) would be readily be able to discern effective combined therapeutic regimens without undue experimentation based on the selected adjunct therapy and the teachings of the instant specification.

In this regard it will be appreciated that the combination of the modified antibody (with or without cytotoxin) and the chemotherapeutic agent may be administered in any order and within any time frame that provides a therapeutic benefit to the patient. That is, the chemotherapeutic agent and modified antibody may be administered in any order or concurrently. In selected embodiments the modified antibodies of the present invention will be administered to patients that have previously undergone chemotherapy. In yet other embodiments, the modified antibodies and the chemotherapeutic treatment will be administered substantially simultaneously or concurrently. For example, the patient may be given the modified antibody while undergoing a course of chemotherapy. In preferred embodiments the modified antibody will be administered within 1 year of any chemotherapeutic agent or treatment. In other preferred embodiments the modified antibody will be administered within 10, 8, 6, 4, or 2 months of any chemotherapeutic agent or treatment. In still other preferred embodiments the modified antibody will be administered within 4, 3, 2 or 1 week of any chemotherapeutic agent or treatment. In yet other embodiments the modified antibody will be administered within 5, 4, 3, 2 or 1 days of the selected chemotherapeutic agent or treatment. It will further be appreciated that the two agents or treatments may be administered to the patient within a matter of hours or minutes (i.e. substantially simultaneously).

Moreover, in accordance with the present invention a myelosuppressed patient shall be held to mean any patient exhibiting lowered blood counts. Those skilled in the art will appreciate that there are several blood count parameters conventionally used as clinical indicators of myelosuppresion and one can easily measure the extent to which myelosuppresion is occurring in a patient. Examples of art accepted myelosuppression measurements are the Absolute Neutrophil Count (ANC) or platelet count. Such myelosuppression or partial myeloablation may be a result of various biochemical disorders or diseases or, more likely, as the result of prior chemotherapy or radiotherapy. In this respect, those skilled in the art will appreciate that patients who have undergone traditional chemotherapy typically exhibit reduced red marrow reserves. As discussed above, such subjects often cannot be treated using optimal levels of cytotoxin (i.e. radionuclides) due to unacceptable side effects such as anemia or immunosuppression that result in increased mortality or morbidity.

More specifically conjugated or unconjugated modified antibodies of the present invention may be used to effectively treat patients having ANCs lower than about 2000/mm³ or platelet counts lower than about 150,000/mm³. More preferably the modified antibodies of the present invention may be used to treat patients having ANCs of less than about 1500/mm³, less than about 1000/mm³ or even more preferably less than about 500/mm³. Similarly, the modified antibodies of the present invention may be used to treat patients having a platelet count of less than about 75,000/mm³, less than about 50,000/mm³ or even less than about 10,000/mm³. In a more general sense, those skilled in the art will easily be able to determine when a patient is myelosuppressed using government implemented guidelines and procedures.

As indicated above, many myelosuppressed patients have undergone courses of treatment including chemotherapy, implant radiotherapy or external beam radiotherapy. In the case of the latter, an external radiation source is for local irradiation of a malignancy. For radiotherapy implantation methods, radioactive reagents are surgically located within the malignancy, thereby selectively irradiating the site of the disease. In any event, the disclosed modified antibodies may be used to treat neoplastic disorders in patients exhibiting myelosuppression regardless of the cause and, specifically, may be used in conjunction with external beam radiation or implant radiotherapy.

In this regard it will further be appreciated that the modified antibodies of the instant invention may be used in conjunction or combination with any chemotherapeutic agent or agents or regimen (e.g. to provide a combined therapeutic regimen) that eliminates, reduces, inhibits or controls the growth of neoplastic cells in vivo. As discussed, such agents often result in the reduction of red marrow reserves. This reduction may be offset, in whole or in part, by the diminished myelotoxicity of the compounds of the present invention that advantageously allow for the aggressive treatment of neoplasms in such patients. In other preferred embodiments the radiolabeled immunoconjugates disclosed herein may be effectively used with radiosensitizers that increase the susceptibility of the neoplastic cells to radionuclides. For example, radiosensitizing compounds may be administered after the radiolabeled modified antibody has been largely cleared from the bloodstream but still remains at therapeutically effective levels at the site of the tumor or tumors.

With respect to these aspects of the invention, exemplary chemotherapic agents that are compatible with the instant invention include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in HARRISON'S PRINCIPLES OF INTERNAL MEDICINE 1774-1788 (Kurt J. Isselbacher et al., eds., 13^{th} ed. 1994) and V. T. DeVita et al., (1997) and the references cited therein for standard dosing and scheduling. These therapies can be used unchanged, or altered as needed for a particular patient, in combination with one or more modified antibodies as described herein.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

For patients with intermediate- and high-grade NHL, who fail to achieve remission or relapse, salvage therapy is used. Salvage therapies employ drugs such as cytosine arabinoside, cisplatin, etoposide and ifosfamide given alone or in combination. In relapsed or aggressive forms of certain neoplastic disorders the following protocols are often used: IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, HD cytarabine, cisplatin), CEPP(B) (cyclophosphamide, etoposide, procarbazine, prednisone and bleomycin) and CAMP (lomustine, mitoxantrone, cytarabine and prednisone) each with well known dosing rates and schedules.

The amount of chemotherapeutic agent to be used in combination with the modified antibodies of the instant invention may vary by subject or may be administered according to what is known in the art. See for example, Bruce A Chabner et al., Antineoplastic Agents, in GOODMAN & GILMAN'S THE PHARMACOLOGICAL BASIS OF THERAPEUTICS 1233-1287 ((Joel G. Hardman et al., eds., 9^{th} ed. 1996).

As previously discussed, the modified antibodies of the present invention, immunoreactive fragments or recombinants thereof may be administered in a pharmaceutically effective amount for the in vivo treatment of mammalian malignancies. In this regard, it will be appreciated that the disclosed antibodies will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of the modified antibody, immunoreactive fragment or recombinant thereof, conjugated or unconjugated to a therapeutic agent, shall be held to mean an amount sufficient to achieve effective binding with selected immunoreactive antigens on neoplastic cells and provide for an increase in the death of those cells. Of course, the pharmaceutical compositions of the present invention may be administered in single or multiple doses to provide for a pharmaceutically effective amount of the modified antibody.

More specifically, they the disclosed antibodies and methods should be useful for reducing tumor size, inhibiting tumor growth and/or prolonging the survival time of tumor-bearing animals. Accordingly, this invention also relates to a method of treating tumors in a human or other animal by administering to such human or animal an effective, non-toxic amount of modified antibody. One skilled in the art would be able, by routine experimentation, to determine what an effective, non-toxic amount of modified antibody would be for the purpose of treating malignancies. For example, a therapeutically active amount of a modified antibody may vary according to factors such as the disease stage (e.g., stage I versus stage IV), age, sex, medical complications (e.g., immunosuppressed conditions or diseases) and weight of the subject, and the ability of the antibody to elicit a desired response in the subject. The dosage regimen may be adjusted to provide the optimum therapeutic response. For instance, several divided doses may be administered daily, or the dose may be proportionally reduced as indicated by the exigencies of the therapeutic situation. Generally, however, an effective dosage is expected to be in the range of about 0.05 to 100 milligrams per kilogram body weight per day and more preferably from about 0.5 to 10, milligrams per kilogram body weight per day.

In keeping with the scope of the present disclosure, the modified antibodies of the invention may be administered to a human or other animal in accordance with the aforementioned methods of treatment in an amount sufficient to produce such effect to a therapeutic or prophylactic degree. The antibodies of the invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of monoclonal antibodies according to the present invention may prove to be particularly effective.

Methods of preparing and administering conjugates of the antibody, immunoreactive fragments or recombinants thereof, and a therapeutic agent are well known to or readily determined by those skilled in the art. The route of administration of the antibody (or fragment thereof) of the invention may be oral, parenteral, by inhalation or topical. The term parenteral as used herein includes intravenous, intraarterial, intraperitoneal, intramuscular, subcutaneous, rectal or vaginal administration. The intravenous, intraarterial, subcutaneous and intramuscular forms of parenteral administration are generally preferred. While all these forms of administration are clearly contemplated as being within the scope of the invention, a preferred administration form would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumine), etc. However, in other methods compatible with the teachings herein, the modified antibodies can be delivered directly to the site of the malignancy site thereby increasing the exposure of the neoplastic tissue to the therapeutic agent.

Preparations for parenteral administration includes sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a modified antibody by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 and U.S. Ser. No. 09/259,338 each of which is incorporated herein by reference. Such articles of manufacture will preferably have labels or package inserts indicating that the associated compositions are useful for treating a subject suffering from, or predisposed to, cancer, malignancy or neoplastic disorders.

As discussed in detail above, the present invention provides compounds, compositions, kits and methods for the treatment of neoplastic disorders in a mammalian subject in need of treatment thereof. Preferably, the subject is a human. The neoplastic disorder (e.g., cancers and malignancies) may comprise solid tumors such as melanomas, gliomas, sarcomas, and carcinomas as well as myeloid or hematologic malignancies such as lymphomas and leukemias. In general, the disclosed invention may be used to prophylactically or therapeutically treat any neoplasm comprising an antigenic marker that allows for the targeting of the cancerous cells by the modified antibody. Exemplary cancers that may be treated include, but are not limited to, prostate, colon, skin, breast, ovarian, lung and pancreatic. In preferred embodiments selected modified antibodies of instant invention (e.g. CC49.ΔC_{H}2) will be used to diagnose or treat colon cancers or other gastric carcinomas. More particularly, the antibodies of the instant invention may be used to treat Kaposi's sarcoma, CNS neoplasms (capillary hemangioblastomas, meningiomas and cerebral metastases), melanoma, gastrointestinal and renal sarcomas, rhabdomyosarcoma, glioblastoma (preferably glioblastoma multiforme), leiomyosarcoma, retinoblastoma, papillary cystadenocarcinoma of the ovary, Wilm's tumor or small cell lung carcinoma. It will be appreciated that appropriate antibodies may be derived for tumor associated antigens related to each of the forgoing neoplasms without undue experimentation in view of the instant disclosure.

Exemplary hematologic malignancies that are amenable to treatment with the disclosed invention include Hodgkins and non-Hodgkins lymphoma as well as leukemias, including ALL-L3 (Burkitt's type leukemia), chronic lymphocytic leukemia (CLL) and monocytic cell leukemias. It will be appreciated that the compounds and methods of the present invention are particularly effective in treating a variety of B-cell lymphomas, including low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL and Waldenstrom's Macroglobulinemia. It should be clear to those of skill in the art that these lymphomas and lukemias will often have different names due to changing systems of classification, and that patients having hematologic malignancies classified under different names may also benefit from the combined therapeutic regimens of the present invention. In addition to the aforementioned neoplastic disorders, it will be appreciated that the disclosed invention may advantageously be used to treat additional malignancies bearing compatible tumor associated antigens.

The foregoing description will be more fully understood with reference to the following examples. Such Examples, are, however, demonstrative of preferred methods of practicing the present invention and are not limiting of the scope of the invention or the claims appended hereto.

### Example 1

### Construction and Expression of a C2B8.ΔC_{H}2 Immunoglobulin

The chimeric antibody C2B8 (IDEC Pharmaceuticals) was modified to create a domain deleted version lacking the C_{H}2 domain within the human gamma 1 constant region. C2B8 and the plasmid N5KG1, which is an “empty” vector encodes a human kappa light chain constant region as well as a human gamma 1 constant region, are described in U.S. Pat. Nos. 5,648,267 and 5,736,137 each of which is incorporated herein by reference. Creation of a C_{H}2 domain deleted version was accomplished by way of overlapping PCR mutagenesis.

The gamma 1 constant domain begins with a plasmid encoded Nhe I site with is in translational reading frame with the immunoglobulin sequence. A 5′ PCR primer was constructed encoding the Nhe I site as well as sequence immediately downstream. A 3′ PCR primer mate was constructed such that it anneals with the 3′ end to the immunoglobulin hinge region and encodes in frame the first several amino acid of the gamma 1 CH3 domain. A second PCR primer pair consisted of the reverse complement of the 3′ PCR primer from the first pair (above) as the 5′ primer and a 3′ primer that anneals at a loci spanning the BsrG I restriction site within the C_{H}3 domain. Following each PCR amplification, the resultant products were utilized as template with the Nhe I and BsrG I 5′ and 3′, respectively primers. The amplified product was then cloned back into N5KG1 to create the plasmid N5KG1ΔC_{H}2. This construction places the intact CH3 domain immediately downstream and in frame with the intact hinge region. As this is an “empty” vector, the C2B8 immunoglobulin light and heavy chain variable domains were then inserted in the appropriate cloning sites.

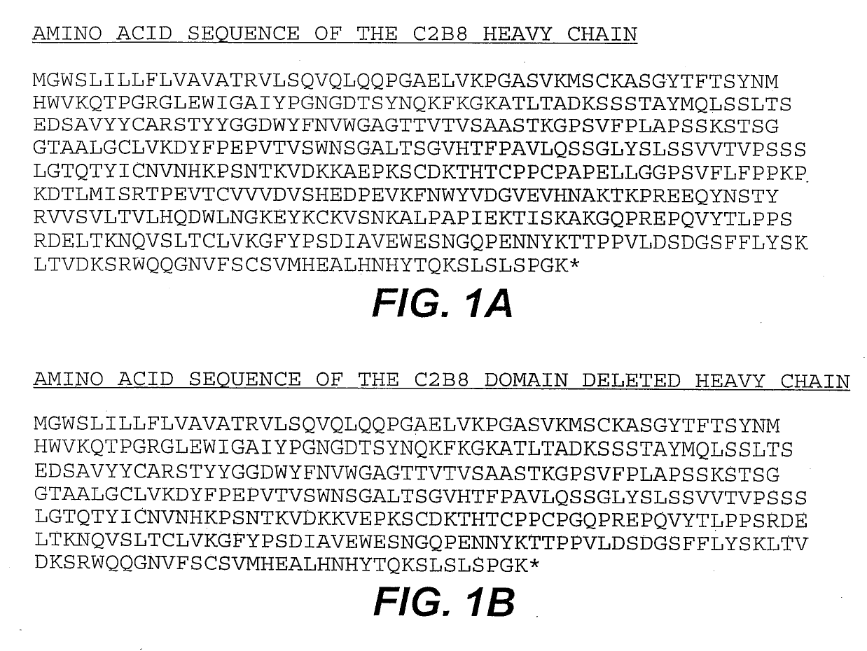

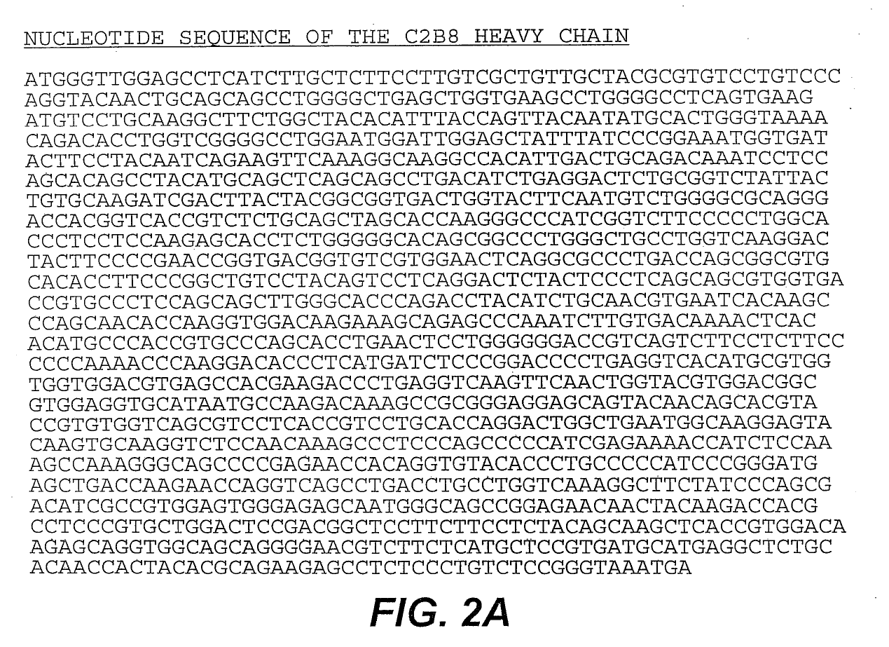

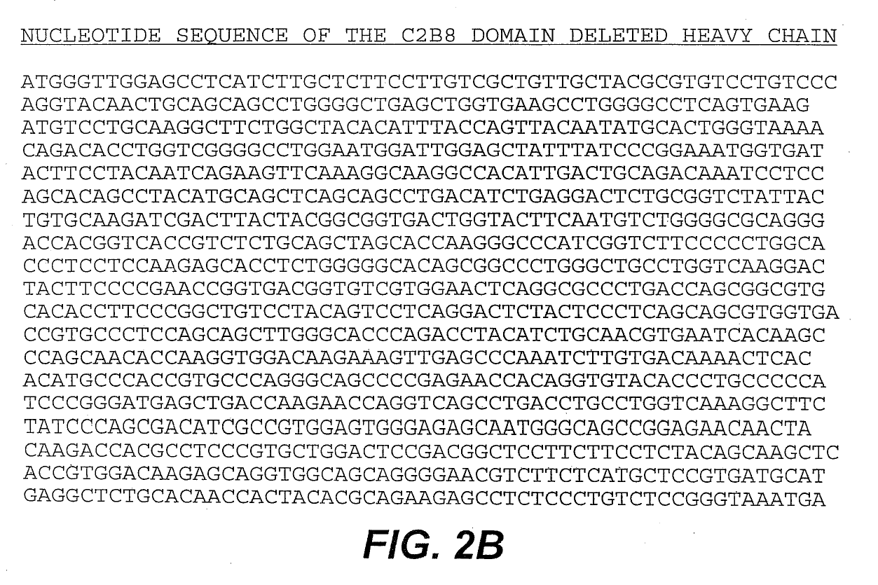

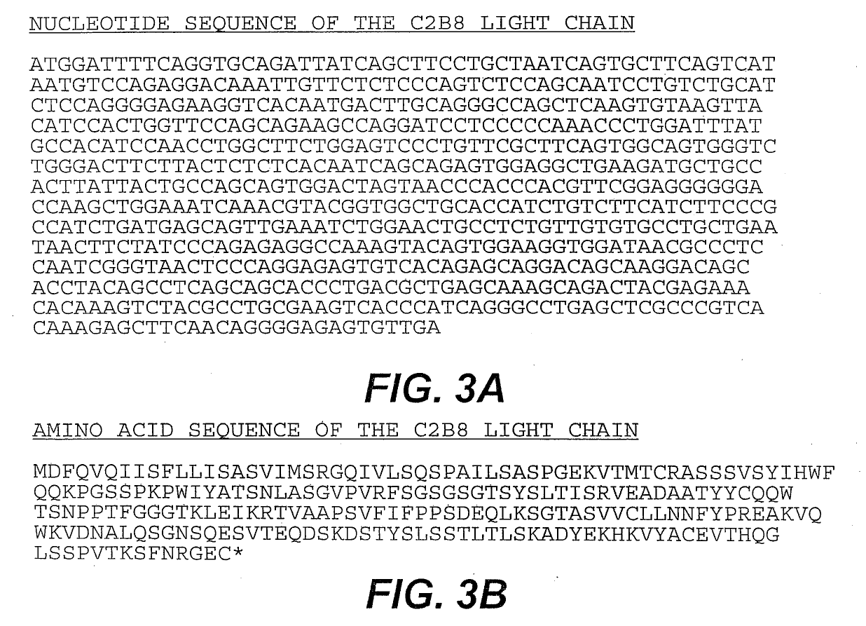

Following sequence confirmation of the immunoglobulin coding regions, this expression construct was transfected into CHO DG44 cells and selected for G418 resistance (conferred by a vector encoded neomycin phosphotransferase gene). Resistant cell isolates were then assayed for HuCC49 immunoglobulin expression. The sequence of the resulting construct is shown in FIGS. 1-3.

### Example 2

### Construction and Expression of a huCC49.ΔC_{H}2 Immunoglobulin

A humanized version of the CC49 antibody (ATCC No. HB 9459) was obtained from the National Cancer Institute. The light chain was encoded in a plasmid referred to as pLNCX II HuCC49 HuK. The Heavy Chain was encoded in a plasmid referred to as pLgpCX II HuCC49G1.ΔC_{H}2.

The light and heavy chain variable domains only were isolated from these plasmids by PCR amplification. PCR primers were constructed such that restriction endonuclease sites were included allowing subsequent subcloning into IDEC's proprietary expression vector N5KG1.ΔC_{H}2.

The light chain restriction enzymes were Bgl II at the 5′ end (immediately upsteam of the translation initiation codon for the natrual leader peptide encoded by the NCI plasmid) and BsiW I at the 3′ end (in translational reading frame with IDEC's vector encoded human kappa light chain constant domain. No amino acids within the light chain variable domain were changed from the NCI sequence.

The heavy chain restriction enzymes were Mlu I at the 5′ end (encoding in frame amino acid residues −5 and −4 of the “synthetic” immunoglobulin heavy chain signal peptide encode by IDEC's expression vector). The PCR primer also encoded residues −3, −2 and −1 with respect to the beginning of the heavy variable domain. The 3′ heavy chain PCR primer encoded the restriction enzyme Nhe I which codes in frame with IDEC's gamma 1 domain deleted heavy chain constant region. The final result is an expression construct encoding the HuCC49 domain deleted antibody with the following components. No amino acids within the heavy chain variable domain were changed from the NCI sequence.

Light chain: Natural light chain leader-NCI variable domain-IDEC's human kappa constant domain.

Heavy chain: IDEC's synthetic heavy leader-NCI variable domain-IDEC's CH2 domain deleted gamma 1 heavy chain constant domain.

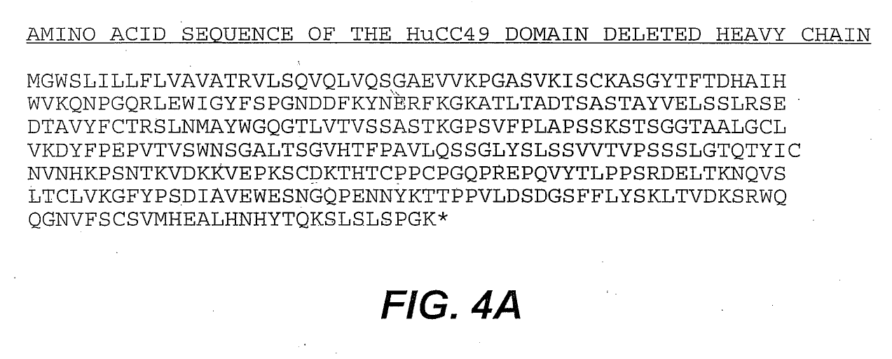

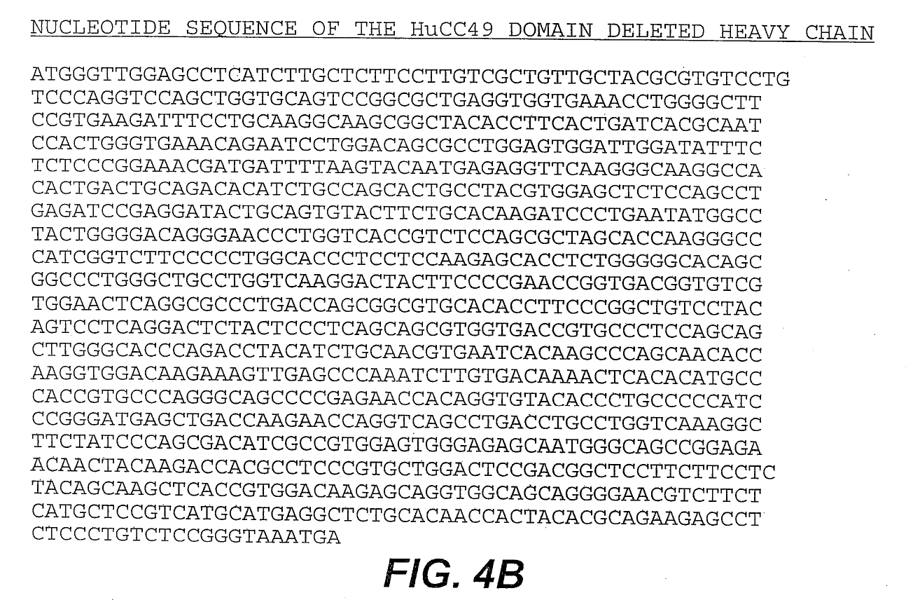

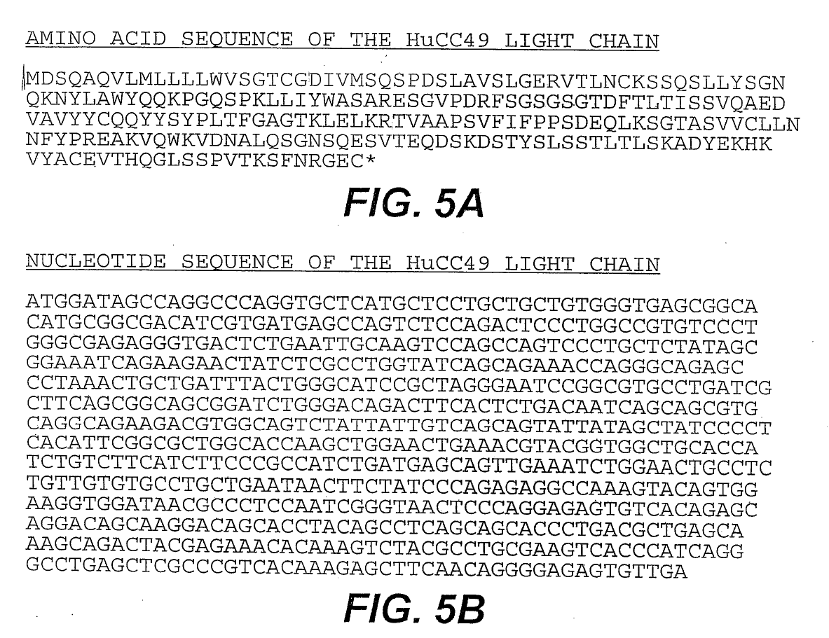

Following sequence confirmation of the immunoglobulin coding regions, this expression construct was transfected into CHO DG44 cells and selected for G418 resistance (conferred by a vector encoded neomycin phosphotransferase gene). Resistant cell isolates were then assayed for HuCC49 immunoglobulin expression. The sequence for huCC49.ΔC_{H}2 heavy and light chains is shown in FIGS. 4 and 5.

### Example 3

### Construction and Expression of a C5E10.ΔC_{H}2 Immunoglobulin

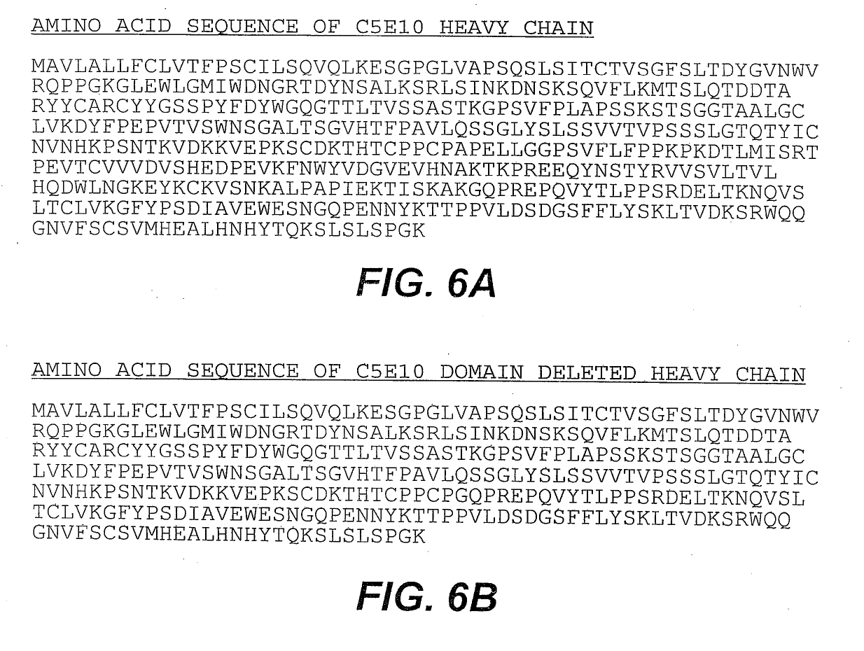

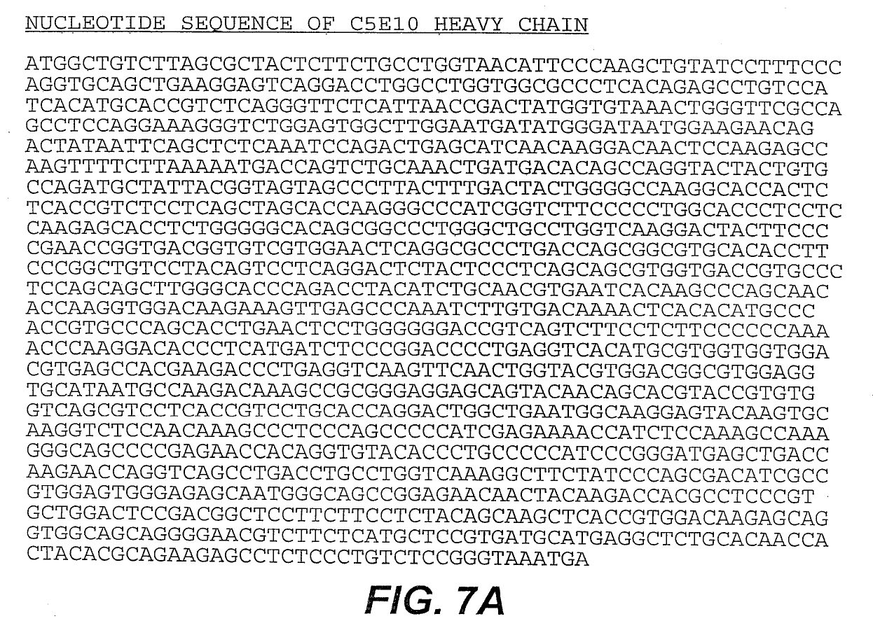

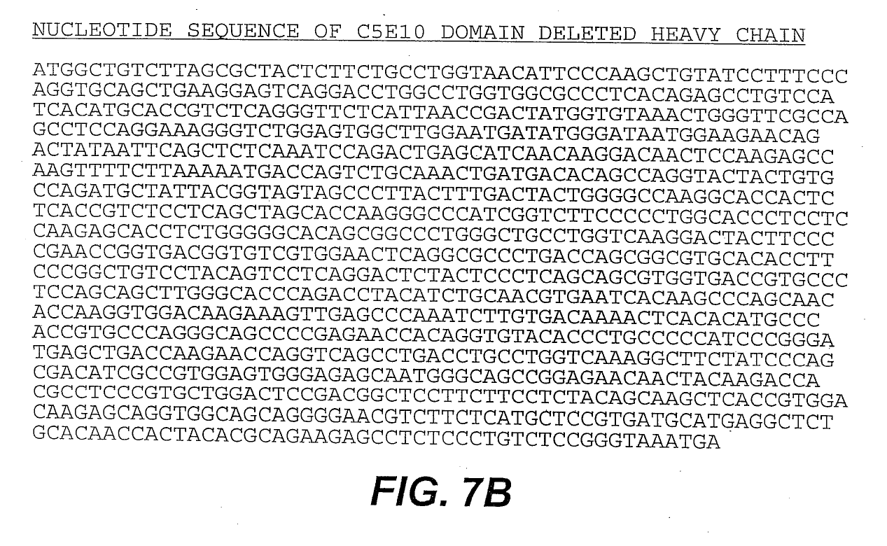

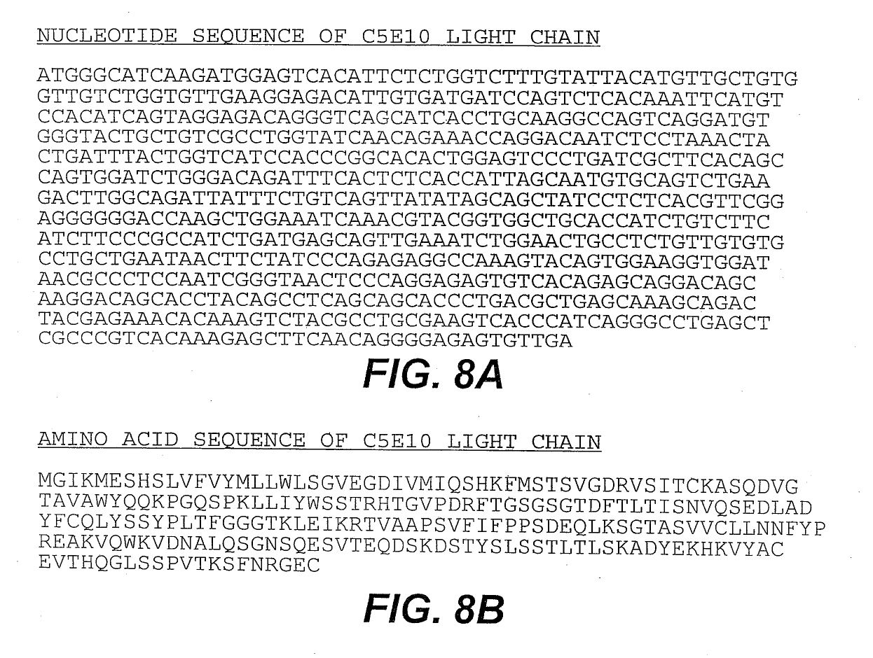

Murine C5E10 expressing hybridoma cells were received from the University of Iowa. RNA from the cells and then made cDNA using oligo dT from the RNA. The cDNA was PCR amplified using a series of mouse kappa and heavy chain variable region primers. The PCR products were run on agarose gels. Using known techniques, primers were used to isolate and identify the light and heavy chains as bands in the agarose. The bands were isolated, cut with restriction enzymes and the light chain variable region was cloned into Neospla N5KG1 vector substantially as described in Examples 1 and 2. The heavy chain variable regions were then cloned into a Neospla ΔC_{H}2 vector (also substantially as described in Examples 1 and 2) in order to generate an antibody missing the C_{H}2 domain. The DNA and amino acid sequences of the heavy and light chain variable regions of the parent antibody and the domain deleted construct were sequenced as shown in FIGS. 6 to 8. The vectors were electroporated into CHO cells using art known techniques to provide for stable cell line development. Following growth of the CHO cells and expression of the product, the modified antibodies were purified using affinity chromatography.

### Example 4

### Prepartion of ¹¹¹In and ⁹⁰Y Radiolabeled Constructs

Modified antibody constructs from Examples 1-3 or substantial equivalents and appropriate controls were labeled with radioactive indium and yttrium for in vivo biodistribution and bioavailability studies as described below. As discussed above, direct incorporation of radioactive metals such as ¹¹¹In and ⁹⁰Y in proteins is not generally effective. As such, chelators are typically used to link these isotopes to the antibody to provide the desired radioactive imunoconjugate. For the studies described herein a MX-DTPA chelator was used to incorporate the ¹¹¹In and ⁹⁰Y.

MAb's 2B8, 2B8.F(ab′)2 and C2B8.ΔC_{H}2 were diafiltered into low metal containing saline (LMC-Saline, pH adjusted to 8.6 using 0.5M Boric acid) before conjugation. The Mabs were diafiltered using pre-washed Centricon 30 filters (two times, according to manufactures instruction), MAb concentration measured by A280 (1 mg/ml=1.7 AU) and diluted using LMC-Saline (pH 8.6) to approximately 10.0 mg/ml. MAb was reacted with MX-DTPA at a 4:1 molar ratio (chelate to MAB) for 14-16 hours at room temperature. After incubation, the conjugate was clarified from unreacted chelate using Centricon 30 filters (3 times), protein concentration determined by A280 and adjusted to a final concentration of 2.0 mg/ml using LMC-Saline.

CC49 and CC49.ΔC_{H}2 were conjugated to MX-DTPA by the same protocol except a 2:1 molar ratio of chelator to MAb was used in place of the 4:1 ratio used for the anti-CD20 MAbs. Antibody concentrations for CC49 and CC49.ΔC_{H}2 were determined by A280 (1 mg/mL=1.0).

Following conjugation, the domain deleted constructs and control antibodies and fragments were radiolabeledt with ¹¹¹In and ⁹⁰Y. The ¹¹¹In were labeled at specific activities ranging from 1 to 3 mCi/mg protein. Indium-[111] chloride in dilute HCl (Nycomed Amersham or Cyclotron Products Inc.) was adjusted to pH 4 using 50 mM sodium acetate. Immunoglobulin conjugate was added and the mixture incubated at ambient temperature. After 30 minutes, the mixture was diluted to a final antibody concentration of 0.2 mg/mL using 1×PBS, pH 7.2 containing 7.5% human serum albumin (HAS) and 1 mM diethylenetriaminepentaacetic acid (DTPA) (formulation buffer).

The constructs and controls were also radiolabeled with ⁹⁰Y at specific activities ranging from 10 to 19 mCi/mg protein. Yttrium-[90] chloride in dilute HCl (Nycomed Amersham or NEN Dupont) was adjusted to pH 4 using 50 mM sodium acetate. Antibody conjugate was added and the mixture incubated at ambient temperature. After 5 minutes, the mixture was diluted to a final antibody concentration of 0.2 mg/mL using 1×PBS, pH 7.2 containing 7.5% human serum albumin (HAS) and 1 mM diethylenetriaminepentaacetic acid (DTPA) (formulation buffer).

### Example 5

### Preparation of ¹²⁵I Radiolabeled Constructs

Constructs from Examples 1-3 and appropriate controls were also labeled with radioactive Iodine for use in the biodistribution and bioavailability studies discussed below. More particularly, the constructs and controls were radiolabeled using Iodo-Beads (BioRad Industries) following the manufacturer's general guidelines. Two mCi of Na¹²⁵I were pre-incubated with one Iodo-Bead for 5 minutes in 100 mM sodium phosphate, pH 7.0. Approximately 0.2 mg of immunoglobulin was added and the reaction mixture incubated for 2 minutes. Unincorporated iodine was removed by desalting on Sephadex G-25 (Pharmacia PD-10 column) into 1×PBS.

### Example 6

### Blood Clearance Rates of Radiolabeled huCC49.ΔC_{H}2

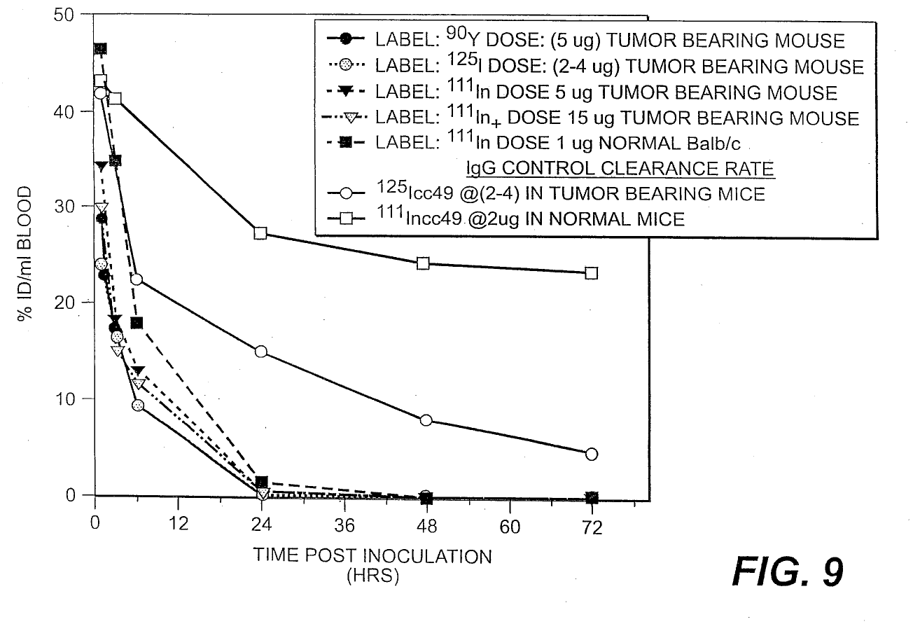

FIG. 9 compares the blood clearance rates of ¹¹¹In, ⁹⁰Y and ¹²⁵I labeled domain deleted huCC49 to ¹¹¹In or ¹²⁵I labeled parent antibody CC49 in mice. The domain deleted constructs or their substantial equivalents and whole antibodies were prepared as described in Examples 1-5. Labeled CC49 constructs were evaluated in either normal mice or LS174T BABL/c nu/nu tumor bearing mice. LS174T is a TAG-72 positive tumor derived from a human colon carcinoma. Tumor xenografts were established and propagated in the mice by sc. injections of 1×10⁶ washed tissue culture cells. As shown in FIG. 9 all domain deleted constructs labeled with the various isotopes exhibited similar clearance rates from the blood in both tumor and nontumor bearing mice. Significantly, it should be noted that greater than 99% of the labeled domain deleted constructs were removed from the blood 24 hours post inoculation. No difference in the clearance rates was observed using the various isotopes. In sharp contrast, significant levels of radiolabeled whole antibodies remained in circulation at greater than three days post injection. As discussed extensively above, the prolonged circulation and nonspecific deposition of the administered radiolabeled compounds can lead to substantial myelotoxicity and, in many cases, actually limit the amount of radioconjugate that may be administered. Rapid clearance of the radioconjugate can drastically reduce this myelotoxicity. Thus, this Example graphically illustrates the advantages of the present invention in reducing undesirable side effects and potentially increasing the dosage of tumorcidal drug that may be administered.

### Example 7

### Comparison of Blood Clearance Rates and Tumor Localization

Murine antibody 2B8 and a chimeric version thereof, C2B8, both react with human CD20 antigen. Pharmacokinetics of serum clearance and tumor localization were examined using 2B8, C2B8.ΔC_{H}2 and 2B8.F(ab′)₂, all labeled with ¹¹¹In, in tumor bearing mice.

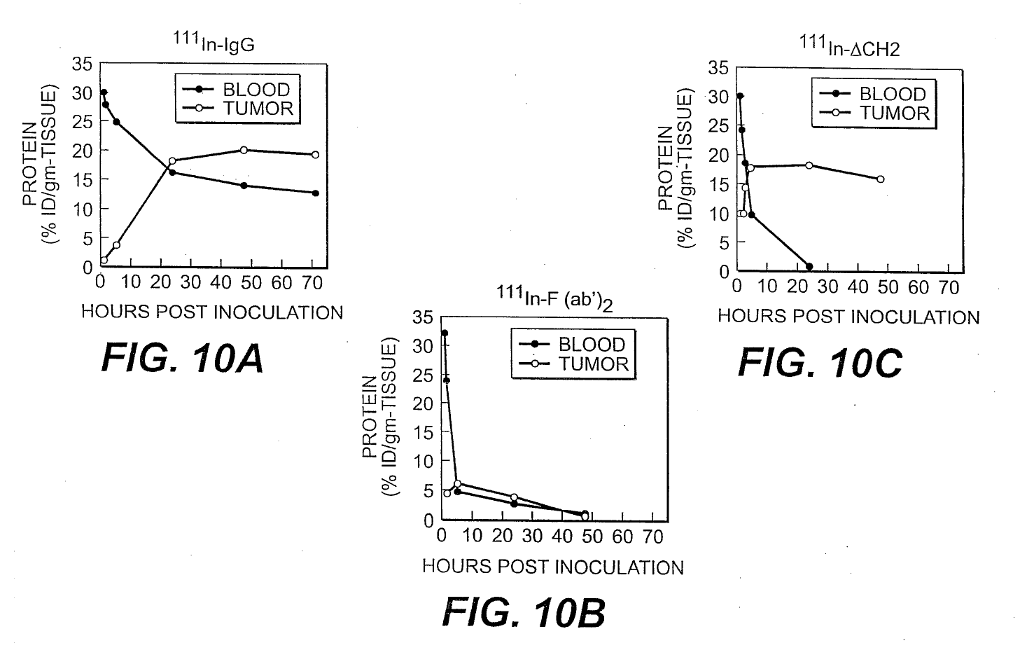

Daudi tumors (CD20 positive) were propagated in female BALB/c nu/nu mice by sc. injections of 1×10⁶ washed tissue culture cells. Radiolabeled Mabs or constructs were injected i.v. when tumor volumes reached a size of approximately 50-100 mm³. For biodistribution and tumor location of the various constructs, animals were sacrificed and bled at the indicated times. In this regard the tumor was removed from the animal, rinsed with PBS and weighed. Standardized blood samples were simply removed stored until analysis. Using art known techniques, radioactivity in the tumor and in the blood was quantified using a gamma counter and corrected for physical decay. Results represent the mean of three animals per time point and are graphically presented in FIG. 10. More specifically, FIG. 10A shows the blood clearance and tumor localization rates for the intact C2B8 while FIGS. 10B and 10C show the same measurements for the labeled F(ab′)2 construct and the domain deleted version respectively.

The curves show that very little of the input radioactivity remained in the circulation 24 hours post infusion using either the ¹¹¹In labeled C2B8.F(ab′)2 (FIG. 10B) or C2B8.ΔC_{H}2 (FIG. 10C) construct. Conversely, relatively high levels of the ¹¹¹In-2B8.IgG remained in the serum 24 hours post infusion (FIG. 10A). Blood clearance rates of both the domain deleted and F(ab′)2 constructs were therefore significantly faster than the intact IgG molecule. More particularly, effective half-lives calculated from the blood clearance rates were 5.7 hours for C2B8.ΔC_{H}2 and 12.9 hours for the 2B8F(ab′)2 fragment compared to 38 hours for the intact 2B8 IgG molecule. The significantly faster blood clearance rate for the domain deleted construct again demonstrates the capacity of the present invention to substantially reduce the radiation dose delivered to the bone marrow.

Conversely, the modified antibodies of the present invention are extremely proficient at delivering therapeutically effective amounts of radioactivity to the tumor itself. In this respect, tumor localization of ¹¹¹In-labeled constructs is also presented in FIG. 10. ¹¹¹In-2B8.IgG showed peak tumor localization 24-48 hours post infusion in FIG. 10A. In contrast, both 2B8.F(ab′)₂ or C2B8.ΔC_{H}2 constructs showed peak localization 6 Hrs post infusion in FIGS. 10B and 10C respectively. However, unlike 2B8.F(ab′)₂ which showed a significant reduction in the percentage injected dose/gm compared to the other constructs, C2B8.ΔC_{H}2 showed tumor localization patterns comparable to amounts obtained using ¹¹¹In2B8 (FIGS. 10A & 10C). In this example, peak tumor localization, expressed as % injected dose per gm tissue (% ID/gm) at 6 hrs using 2B8.F(ab′)2 was 6.2, whereas the domain deleted version at 6 hours was 17.1%. In contrast, 6 hrs only 4% of the 2B8.IgG localized in the tumor. The highest peak localization for 2B8.IgG was at 24 hours and was 19.4%.

Thus, only the modified antibodies of the present invention exhibit the desirous characteristics of high tumor localization combined with relatively quick blood clearance. More generally, the intact antibodies appear to provide for relatively high tumor localization (although after a prolonged period) but are fairly myelotoxic due to an extended blood half-life. Conversely, the F(ab′)2 constructs exhibit relatively quick blood clearance but extremely poor tumor localization. It will be appreciated these limitations are surprisingly overcome by the modified antibodies disclosed herein.

### Example 8

### Examination of Blood Clearance Rates and Tumor Localization

The effective half-lives of the constructs and the MIRD dose estimate radiation to the bone marrow were calculated from the blood clearance data and is shown below in Table 1. Tumor localization data of the immunoconjugates is shown in Table 2. The reported doses were injected i.v. into BALB/c nu/nu mice exhibiting the appropriate tumor (i.e. Daudi or LS174T mice from Examples 6 and 7) and blood was harvested at preselected time points.

Those skilled in the art will appreciate that MIRD (absorbed radiation) dose estimates to the bone marrow were calculated from the percentage-inoculated dose per gm tissue (% ID/gm) using samples taken from 1 to 72 hours post infusion and are reported in Table 1.

TABLE 1
Comparison of Dose Related Parameters for Y2B8 (IgG and
F(ab)2] and CH2 Domain Deleted Constructs for
Normal Tissue (Blood and Red Marrow)

| | | | Dose | | Residence | | Dose |
|---|---|---|---|---|---|---|---|
| | | | In- | Effective | Time | MIRD | Factor- |
| | | | jected | T½-life | (uCi-hr/ | (rad/ | IgG |
| Mab | Type | Label | (ug) | (hrs) | uCi) | mCi) | Ratio |
| CC49 | ΔC_{H}2 | ¹¹¹In | 5 | 5.7 | 0.25 | 0.6 | −3.7 |
| CC49 | ΔC_{H}2 | ¹¹¹In | 10 | 6.5 | 0.27 | 0.61 | −3.7 |
| 2B8 | F(ab)2 | ¹¹¹In | 10 | 12.9 | 0.31 | 0.71 | −3.1 |
| 2B8 | IgG | ¹¹¹In | 10 | 38 | 0.97 | 2.2 | 1.0 |

An examination of Table 1 reaffirms that the domain deleted constructs provide for substantially shorter half-lives and for correspondingly lower doses of radiation to the marrow. More specifically, Table 1 shows that the F(ab′)2 C2B8 construct and the intact IgG had half-lives of 12.9 hours and 38 hours respectively. In sharp contrast the domain deleted CC49 construct only had a half-life of 6.5 hours at the same dose (i.e. more than 5 times less that the intact IgG). Significantly, this short half life leads to substantially less exposure of the blood and red marrow to undesirable radioactive energy. A review of the MIRD levels (essentially radioactive energy delivered to the marrow) shows that the intact C2B8 IgG gave a dose of almost 4 times that provided by the same amount of domain deleted CC49 (i.e. 2.2. rad/mCi vs 0.61 rad/mCi). It should be emphasized that this reduction in marrow exposure will lead to considerably less myelotoxicity, a critical factor in developing therapeutic regimens for cancer treatment.

As indicated above, Table 2 shows the advantages of the present invention in providing for high tumor localization of the radionuclide. It will be appreciated that this enhanced localization, combined with the rapid blood clearance demonstrated above, allows for the particularly effective administration of radioactive or cytotoxic compounds to the site of the neoplastic cells.

TABLE 2
Comparative Dosimetry of Y2B8 [IgG and F(ab)₂] to huCC49ΔCH2
In Tumor Bearing Nude Mouse Xenografts (Tumor Localization)

| | | | Dose | Peak Tumor | Residence | Tumor | |
|---|---|---|---|---|---|---|---|
| | | | Injected | Localization | Time | Dose Factor | Dose Factor- |
| Mab | Type | Label | (ug) | (% ID/gm) | (uCi-hr/uCi) | (rad/mCi) | IgG Ratio |
| CC49 | ΔCH2 | ¹²⁵I | 2 | 16.2% | 0.92 | 3095 | 2.3 |
| CC49 | ΔCH2 | ¹¹¹In | 5 | 17.8% | 1.15 | 3637 | 2.7 |
| 2B8 | F(ab)2 | ¹¹¹In | 10 | 5.5% | 0.65 | 618 | −2.1 |
| 2B8 | IgG | ¹¹¹In | 10 | 18.5% | 0.95 | 1331 | 1.0 |

As shown in Table 2, ¹¹¹In-2B8; ¹¹¹In-huCC49.ΔC_{H}2 and ¹²⁵I-huCC49.ΔC_{H}2 showed similar tumor residence times (0.95, 1.15 and 0.92 uCi-hr/uCi respectively). Additionally, peak localization of ¹¹¹In-huCC49.ΔC_{H}2, ¹²⁵I-huCC49.ΔC_{H}2 and ¹¹¹In-2B8 (18.5, 16.2, and 17.8% ID/gm, respectively) was also similar, but peaked at 6 hours post infusion for the domain deleted constructs compared to 24 hours post inoculation for the intact 2B8. The earlier localization of domain deleted constructs (using either ¹¹¹In or ¹²⁵I labeled fragments) resulted in a estimated 3 fold increase in the radiation dose to the tumor when compared to the intact parent MAb, 2B8 (i.e. 3637 rad/mCi vs 1331 rad/mCi).

Again, it should be emphasized that the faster blood clearance and increased tumor targeting without compromising either peak tumor localization or tumor retention time demonstrated using domain deleted constructs represents a significant advantage for clinical protocols using combination drug therapy.

### Example 9

### Synergistic Properties of Modified Antibodies

Forty athymic female mice were injected subcutaneously with 0.2 mL of 2×10⁶ LS174T cells. The TAG-72⁺ tumors were allowed to grow to a palable size of 150-200 mm³. At this time the mice were separated in to four groups of 10 mice each. The four groups were treated as follows:

1. Etoposide alone

2. ⁹⁰Y-huCC49.ΔC_{H}2 alone.

3. ⁹⁰Y-huCC49.ΔC_{H}2+etoposide

4. Diluent control (PBS/DMSO)

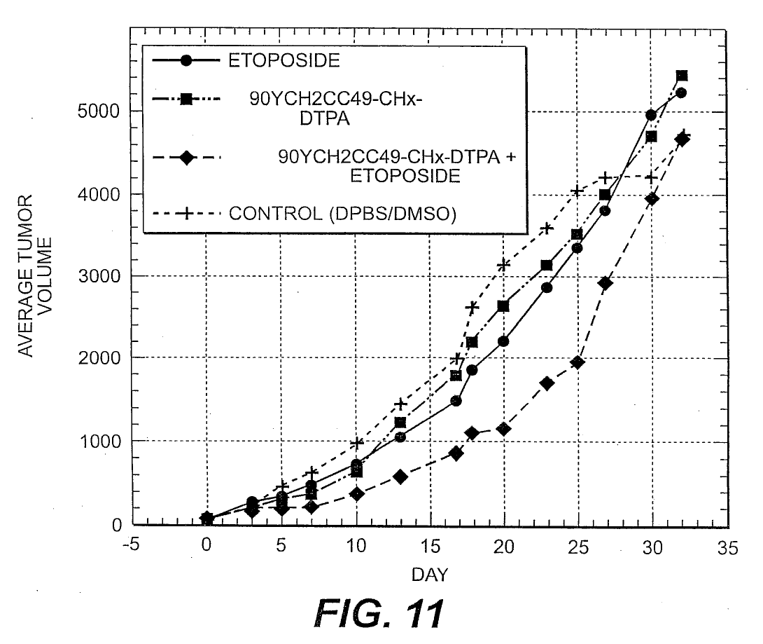

More particularly, a stock solution of etoposide was made, at 100 mg/mL in DMSO. This was then diluted to 6.88 mg/mL in PBS. In group 1 the mice were injected with 1.72 mg of etoposide, repeated every fourth day, for a total of three injections. In group 2, the mice were injected with 0.05 of mCi of ⁹⁰Y-huCC49.ΔC_{H}2 using a CHx-DTPA chelator to affix the radioisotope. In group 3, the mice were injected with 0.05 mCi of the same radiolabeled modified antibody and 1.72 mg of etoposide followed by two later injections of 1.72 mg of etoposide. The control group mice (4) were injected with PBS/DMSO, at a concentration of 6.9% DMSO every fourth day for a total of three injections. The tumors were measured two or three times per week and graphically illustrated FIG. 11.

FIG. 11 shows that the combination of etoposide along with the domain deleted radiolabled CC49 antibody retards the growth of tumor mass more than either agent alone. This synergistic result is particularly evident at day 25 where the tumor burden is reduced by almost half through the use of the combination of the agents when compared to either the mice treated with ⁹⁰Y-huCC49.ΔC_{H}2 or etoposide.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1. A domain deleted CC49 antibody reactive with TAG-72 comprising a heavy chain having an amino acid sequence substantially as set forth in FIG. 4A.

2. The domain deleted CC49 antibody of claim 1 further comprising a cytotoxic agent.

3. The domain deleted CC49 antibody of claim 2 wherein said cytotoxic agent is a radionuclide.

4-5. (canceled)

6. The domain deleted CC49 antibody of claim 1 further comprising an amino acid spacer.

7. The a domain deleted CC49 antibody of claim 1, having a heavy chain amino acid sequence substantially as set forth in FIG. 4A covalently linked to one or more bifunctional chelators wherein said one or more bifunctional chelators is associated with 90Y.

8. The domain deleted CC49 antibody of claim 7 wherein said bifunctional chelator is selected from the group consisting of MX-DTPA and CHX-DTPA.

9. A domain deleted C2B8 antibody reactive with CD20 comprising a heavy chain having an amino acid sequence substantially as set forth in FIG. 1B.

10. The domain deleted C2B8 antibody of claim 9 further comprising a cytotoxic agent.

11. The domain deleted C2B8 antibody of claim 10 wherein said cytotoxic agent is a radionuclide.

12.-13. (canceled)

14. A method of imaging a neoplasm comprising a tumor associated antigen in a patient in need thereof comprising the steps of: administering a modified antibody to said patient wherein said modified antibody is associated with an imaging agent and binds to said tumor associated antigen; and imaging said patient to reveal said neoplasm.

15. The method of claim 14 wherein said imaging agent is a radioisotope.

16. The method of claim 15 wherein said radioisotope is associated with said modified antibody via a bifunctional chelator.

17. (canceled)

18. A method of treating a patient suffering from a neoplastic disorder comprising the step of administering a therapeutically effective amount of a modified antibody to said patient.

19. The method of claim 18 wherein said modified antibody comprises a domain deleted antibody.

20. (canceled)

21. The method of claim 19 wherein said domain deleted antibody comprises an amino acid spacer.

22. The method of claim 18 wherein said modified antibody reacts with a tumor associated antigen.

23-25. (canceled)

26. The method of claim 18 wherein said modified antibody is associated with a cytotoxic agent.

27. The method of claim 26 wherein said cytotoxic agent comprises a radioisotope.

28-29. (canceled)

30. The method of claim 18 wherein said neoplastic disorder is a hematologic neoplasm.

31. The method of claim 18 wherein said myelosuppressed patient exhibits an ANC of less than about 1500/mm3.

32. The method of claim 31 wherein said myelosuppressed patient has a white cell count of less than about 1000/mm3.

33. A method of treating a patient exhibiting a neoplastic disorder comprising the steps of: administering a therapeutically effective amount of at least one chemotherapeutic agent to said patient; and administering a therapeutically effective amount of at least one modified antibody to said patient wherein said chemotherapeutic agent and said modified antibody may be administered in any order or concurrently.

34. The method of claim 33 wherein said modified antibody comprises a domain deleted antibody.

35. (canceled)

36. The method of claim 33 wherein said domain deleted antibody comprises a spacer.

37. The method of claim 33 wherein said modified antibody reacts with a tumor associated antigen.

38-40. (canceled)

41. The method of claim 33 wherein said modified antibody is associated with a cytotoxic agent.

42. The method of claim 41 wherein said cytotoxic agent comprises a radioisotope.

43-44. (canceled)

45. The method of claim 33 wherein said neoplastic disorder is a hematologic neoplasm.

46. The method of claim 33 wherein said patient has a white cell count of less than about 1500/mm3.

47. The method of claim 33 wherein said patient has a white cell count of less than about 1000/mm3.

48. The method of claim 33 wherein said chemotherapeutic agent is administered prior to said modified antibody.

49. The method of claim 48 wherein said modified antibody is administered within a month of said chemotherapeutic agent.

50. The method of claim 48 wherein said modified antibody is administered within two weeks of said chemotherapeutic agent.

51-60. (canceled)

61. The method of claim 18 wherein the patient is myelosuppressed.

62. The method of claim 18 wherein the patient is currently undergoing a course of chemotherapy.

63. The method of claim 18 wherein the patient is a relapsed patient.

64. The method of claim 18 wherein neoplasm is color coded.

65. The method of claim 18 wherein modified antibody is selected from the group consisting of CC49.ΔCH2 AND C2B8.ΔCH2.
